SEQUENCE LISTING (14 sequences)

SEQ ID NO: 1 - DNA, 5508 bp, Homo sapiens
agaagcgggccagcgccgccgggaaaggaggtcgccgcccggggtcgcccgggcttgcgggtcgccgcgcttcctctgcgggcgctgcgggaggcgagggccgcctcggcggaggaagccgaccccggggtctccgccagcccgccggcgccgcctccgagccgcctcggccgtagcctcggcccgggcgggaggcggcggccgccggcgaggcgaggcggcccgcgccctgcctgtcaggccaccggcccggcccgcgggctgcccccgatgcggaggcgctgtcgccggggccatgcagcaggcgccgcagccttacgagttcttcagcgaggagaacagtccgaaatggcggggactgttggtctcggccctgcggaaggttcaggaacaagtgcatcccactctctcagctaatgaagagtctctctattatattgaagagctgatttttcagctgcttaataaattatgcatggcccagccaaggactgttcaagatgtagaggagcgagttcagaagacctttcctcacccaattgataaatgggccattgctgatgcacaatctgctatagaaaaacgaaaacgaagaaatcctcttttactgcctgtggacaaaatccatccttcgttgaaggaagtattagggtacaaagtggactaccatgtatccctatatattgtggctgtactagagtatatctcagctgatattttaaaattggctggtaattatgtttttaatatccggcattatgaaatatctcagcaggacattaaagtgtcaatgtgtgcggataaggttttgatggacatgtttgatcaggatgacataggtttggtttctctctgtgaagatgaacctagttcttctggtgaattaaactactatgatcttgtcagaactgaaatcgcagaagaaagacagtatctacgggaattaaatatgatcataaaagtgtttcgagaagcctttctttctgatagaaagctgtttaaaccttctgatatcgaaaagatttttagtaacatttcagatatacatgaattgactgtgaaacttttaggtttgattgaagacacagttgaaatgactgatgaaagcagtcctcatcccttagctggcagctgttttgaagatttggcagaagagcaagcatttgatccttatgaaacattatcacaggacattctttcaccagagtttcatgaacatttcaataaattgatggccagacctgcagttgctctacactttcagtccattgctgatggttttaaagaggcagttcgttatgtccttccacgtcttatgctggtgccagtgtatcactgttggcactactttgagttactaaagcaattgaaagcatgtagtgaagaacaagaagacagagaatgtttgaaccaagctattactgctctcatgaatctccaaggtagcatggaccgaatttacaagcagtattcacctagacgtcgacctggagatcctgtttgccctttttatagtcaccaattaagaagcaaacacctggctatcaaaaaaatgaatgaaattcagaaaaatatcgatggatgggaaggcaaagatattggacagtgttgtaatgaattcattatggagggaccattgacaagaatcggtgccaaacatgaacggcatatttttctgtttgatggcttaatgatcagttgtaaacctaatcatggccagactcggcttccaggttacagtagtgcagaatacaggttaaaagaaaaatttgtcatgaggaaaatacaaatttgtgataaagaagatacttgtgagcacaagcatgcatttgaattagtatccaaagatgagaacagcataatatttgctgctaagtctgctgaagaaaaaaacaactggatggcagcccttatttctcttcattatcgtagtactctagatcgaatgttagattcagtattattgaaagaagaaaatgagcaaccactgagattaccaagtcctgaagtatatcgttttgtagtaaaagactctgaggaaaacattgtttttgaagacaacttgcaaagtagaagtggcatccccattattaaaggaggaactgtagtgaaattaattgaaaggttaacatatcatatgtatgcagatcccaattttgttcgtacttttcttaccacatatcgttcattttgtaaaccacaggaattgctgagcttactgattgaacggtttgaaattccagagccagaacctactgacgcagacaaattggcaatagagaaaggcgagcagccaatcagtgcagaccttaaaagatttcgcaaggaatatgtccaaccagtacaacttaggatcttaaatgtatttcggcattgggttgaacatcatttttatgactttgaaagagacttggaattgcttgaaagactagaatccttcatttcaagtgtaagagggaaagctatgaaaaaatgggtagagtcaattgctaagatcatcaggaggaagaagcaagctcaggcaaacggagtaagccataatattacctttgaaagtccacctccaccaattgaatggcatatcagcaaaccaggacagtttgaaacatttgatctcatgacacttcatccaatagaaattgcacgtcagctgacacttttggagtctgatctttacaggaaagttcaaccgtctgaacttgtagggagtgtgtggaccaaagaagataaagaaataaattctccaaatttattaaaaatgattcgccataccacaaatctcaccctctggtttgaaaaatgcattgtggaagcagaaaattttgaagaacgggtggcagtactaagtagaattatagaaattctgcaagtttttcaagatttgaataatttcaatggcgtattggagatagtcagtgcagtaaattcagtgtcagtatacagactagaccatacctttgaggcactgcaggaaaggaaaaggaaaattttggacgaagctgtggaattaagtcaagatcactttaaaaaatacctagtaaaacttaagtcaatcaatccaccttgtgtgcctttttttggaatatatttaacaaatattctgaagaccgaagaagggaataatgattttttaaaaaagaaagggaaagatttaatcaatttcagtaagaggaggaaagtagctgaaattactggagaaattcagcagtatcagaatcagccttactgtttacggatagaaccagatatgaggagattctttgaaaaccttaaccccatgggaagtgcatctgaaaaagagtttacagattatttgttcaacaagtcactagaaattgaacctcgaaactgcaaacagccacctcgatttcctaggaaatcaactttttccttaaaatctcctggaataaggcctaacacaggccgacatggctctacctcaggtactttacgaggtcacccaacaccattagaaagagaaccatgtaaaataagctttagtcggattgctgaaactgagctggaatcaacagtgtcagcaccaacctctccaaatacaccatctactccaccagtatctgcttcttcagaccttagtgtatttttagatgtggatctcaacagctcctgtggcagcaatagcatctttgctccagtgcttttgccacattcaaagtctttctttagttcatgtggtagtttacataaactaagtgaagagcccctgattcctcctcctcttcctcctcgaaaaaagtttgatcatgatgcttcaaattccaagggaaatatgaaatctgatgatgatcctcctgctattccaccgagacagcctcctcctccaaaggtaaaacccagagttcctgttcctactggtgcatttgatgggcctctgcatagtccacctccgccaccaccaagagatcctcttcctgatacccctccaccagttccccttcggcctccagaacactttataaactgtccatttaatcttcagccacctccactggggcatcttcacagagattcagactggctcagagacattagtacgtgtccaaattcgccaagcactcctcctagcacaccctctccaagggtaccgcgtcgatgctatgtgctcagttctagtcagaataatcttgctcatcctccagctccccctgttccaccaaggcagaattcaagccctcatctgccaaaactgccaccaaagacttacaaacgggagctttcgcaccccccattgtacagactgcctttgctagaaaatgcagaaactccccaatgaccttagccatatgtagtcattgacactggaatggtatttgtaaagttttttttttaatttattcaaaaaaagacatagtattttagtactttttacaaataatgctcttacaaaaatgctactgatcaaataagcttttaagaattggaaaaataaaaatacaaaagtccttcaccattatcctcaggtgatcagtagcattgccttgtcttggatcctcagtgctgccaaaaggccagtataaagaatttatatttgcactgtaaactctgcaaaaatatggtttaaagtgacatgattgcactgaaaagggatagtgcttttgtgaaatttttcaaatttgagtaatagatgcctttttaaggcagtgaatttacacaaatatgggaggggtatatggtgttactgatttttaaacctctttgaccatcttctagtttttacttctagtttttacatctaggagaattgtgaataacaccaactgttcttaaactctttaatgccatgtcttaaatgccggtatttgctgctgaagacaaaaatgaaaagtaggatgaaaataatagaatggcactgtaagtgtttattattttgtcaaaatgtaaacaaagactacataacccaatgatggagggaaaaagggcatgtatctcattcagatgtgccttttgtttttgcagactatgacgtctttagctaatgaattgcctattgttatggaaaacagttaatatgccatgtatgtacagttttgtttatattgtatatttaaagatactgctaataacctatataaatttaagtgacttgaggcctataatacaatctgctactttactaattcataaattcaaaaaaaattctatggcataggaaccaactgccttgccttcaagacctagtaactttctctataaatctcgtgttaactgaaattttttttaaatatattttttagattggtaatatttaaaccagcaaatacttaaagctttattaaacattttaatcagataagtgagtaaagcttttatttgccatttggatgccttcgttcaaagtgatagagtgttttgctgatagtgctgtagcagcagttgtaaagtagccaaaagccacgttgtttatttactggtttgtggccttttactgtgctttgtatcagagttcttaacaagattaataaatcaccccagtcttaatttttaaaa

SEQ ID NO: 2 - RNA, 21 nt, Artificial Sequence
  Description of Artificial Sequence Synthetic oligonucleotide
  modified_base (1)..(1): 2′ fluoro-modified nucleoside
  misc_feature (1)..(2): Phosphorothioate linkage
  modified_base (2)..(2): 2′ O-methyl modified nucleoside
  misc_feature (2)..(3): Phosphorothioate linkage
  modified_base (3)..(3): 2′ fluoro-modified nucleoside
  modified_base (4)..(4): 2′ O-methyl modified nucleoside
  modified_base (5)..(5): 2′ fluoro-modified nucleoside
  modified_base (6)..(6): 2′ O-methyl modified nucleoside
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′ O-methyl modified nucleoside
  modified_base (11)..(11): 2′ fluoro-modified nucleoside
  modified_base (12)..(12): 2′ O-methyl modified nucleoside
  modified_base (13)..(13): 2′ fluoro-modified nucleoside
  modified_base (14)..(14): 2′ O-methyl modified nucleoside
  modified_base (15)..(15): 2′ fluoro-modified nucleoside
  modified_base (16)..(16): 2′ O-methyl modified nucleoside
  modified_base (17)..(17): 2′ fluoro-modified nucleoside
  modified_base (18)..(18): 2′ O-methyl modified nucleoside
  modified_base (19)..(19): 2′ fluoro-modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 3 - RNA, 21 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ O-methyl modified nucleoside
  misc_feature (2)..(3): Phosphorothioate linkage
  modified_base (3)..(3): 2′ O-methyl modified nucleoside
  modified_base (4)..(4): 2′ O-methyl modified nucleoside
  modified_base (5)..(5): 2′ fluoro-modified nucleoside
  modified_base (6)..(6): 2′ O-methyl modified nucleoside
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′ O-methyl modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ O-methyl modified nucleoside
  modified_base (15)..(15): 2′ O-methyl modified nucleoside
  modified_base (16)..(16): 2′ O-methyl modified nucleoside
  modified_base (17)..(17): 2′ O-methyl modified nucleoside
  modified_base (18)..(18): 2′ O-methyl modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 4 - RNA, 21 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ O-methyl modified nucleoside
  misc_feature (2)..(3): Phosphorothioate linkage
  modified_base (3)..(3): 2′ O-methyl modified nucleoside
  modified_base (4)..(4): 2′ O-methyl modified nucleoside
  modified_base (5)..(5): 2′ fluoro-modified nucleoside
  modified_base (6)..(6): 2′ O-methyl modified nucleoside
  modified_base (7)..(7): 2′ fluoro-modified nucleoside
  modified_base (8)..(8): 2′ O-methyl modified nucleoside
  modified_base (9)..(9): 2′ fluoro-modified nucleoside
  modified_base (10)..(10): 2′ O-methyl modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ O-methyl modified nucleoside
  modified_base (15)..(15): 2′ O-methyl modified nucleoside
  modified_base (16)..(16): 2′ O-methyl modified nucleoside
  modified_base (17)..(17): 2′ O-methyl modified nucleoside
  modified_base (18)..(18): 2′ O-methyl modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 5 - RNA, 22 nt, Artificial Sequence
  Description of Artificial Sequence Synthetic oligonucleotide
  modified_base (1)..(1): 2′ fluoro-modified nucleoside
  misc_feature (1)..(2): Phosphorothioate linkage
  modified_base (2)..(2): 2′ O-methyl modified nucleoside
  misc_feature (2)..(3): Phosphorothioate linkage
  modified_base (3)..(3): 2′ fluoro-modified nucleoside
  modified_base (4)..(4): 2′ O-methyl modified nucleoside
  modified_base (5)..(5): 2′ fluoro-modified nucleoside
  modified_base (6)..(6): 2′ O-methyl modified nucleoside
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′ O-methyl modified nucleoside
  modified_base (11)..(11): 2′ fluoro-modified nucleoside
  modified_base (12)..(12): 2′ O-methyl modified nucleoside
  modified_base (13)..(13): 2′ fluoro-modified nucleoside
  modified_base (14)..(14): 2′ O-methyl modified nucleoside
  modified_base (15)..(15): 2′ fluoro-modified nucleoside
  modified_base (16)..(16): 2′ O-methyl modified nucleoside
  modified_base (17)..(17): 2′ fluoro-modified nucleoside
  modified_base (18)..(18): 2′ O-methyl modified nucleoside
  modified_base (19)..(19): 2′ fluoro-modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
  modified_base (22)..(22): a, c, u, g, unknown or other
  See specification as filed for detailed description of substitutions and preferred embodiments
nnnnnnnnnnnnnnnnnnnnnn

SEQ ID NO: 6 - RNA, 22 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ O-methyl modified nucleoside
  misc_feature (2)..(3): Phosphorothioate linkage
  modified_base (3)..(3): 2′ O-methyl modified nucleoside
  modified_base (4)..(4): 2′ O-methyl modified nucleoside
  modified_base (5)..(5): 2′ fluoro-modified nucleoside
  modified_base (6)..(6): 2′ O-methyl modified nucleoside
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′ O-methyl modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ O-methyl modified nucleoside
  modified_base (15)..(15): 2′ O-methyl modified nucleoside
  modified_base (16)..(16): 2′ O-methyl modified nucleoside
  modified_base (17)..(17): 2′ O-methyl modified nucleoside
  modified_base (18)..(18): 2′ O-methyl modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
  modified_base (22)..(22): a, c, u, g, unknown or other
  See specification as filed for detailed description of substitutions and preferred embodiments
nnnnnnnnnnnnnnnnnnnnnn

SEQ ID NO: 7 - RNA, 21 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′ O-methyl modified nucleoside
  modified_base (4)..(4): 2′ fluoro-modified nucleoside
  modified_base (5)..(5): 2′ O-methyl modified nucleoside
  modified_base (6)..(6): 2′ fluoro-modified nucleoside
  modified_base (7)..(7): 2′ O-methyl modified nucleoside
  modified_base (8)..(8): 2′ fluoro-modified nucleoside
  modified_base (9)..(9): 2′ O-methyl modified nucleoside
  modified_base (10)..(10): 2′ fluoro-modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ fluoro-modified nucleoside
  modified_base (15)..(15): 2′ O-methyl modified nucleoside
  modified_base (16)..(16): 2′ fluoro-modified nucleoside
  modified_base (17)..(17): 2′ O-methyl modified nucleoside
  modified_base (18)..(18): 2′ fluoro-modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 8 - RNA, 21 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′ O-methyl modified nucleoside
  modified_base (4)..(4): 2′ O-methyl modified nucleoside
  modified_base (5)..(5): 2′ O-methyl modified nucleoside
  modified_base (6)..(6): 2′ fluoro-modified nucleoside
  modified_base (7)..(7): 2′ O-methyl modified nucleoside
  modified_base (8)..(8): 2′ fluoro-modified nucleoside
  modified_base (9)..(9): 2′ fluoro-modified nucleoside
  modified_base (10)..(10): 2′ O-methyl modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ fluoro-modified nucleoside
  modified_base (15)..(15): 2′ O-methyl modified nucleoside
  modified_base (16)..(16): 2′ fluoro-modified nucleoside
  modified_base (17)..(17): 2′ O-methyl modified nucleoside
  modified_base (18)..(18): 2′ O-methyl modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 9 - RNA, 21 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′ O-methyl modified nucleoside
  modified_base (4)..(4): 2′ O-methyl modified nucleoside
  modified_base (5)..(5): 2′ O-methyl modified nucleoside
  modified_base (6)..(6): 2′ fluoro-modified nucleoside
  modified_base (7)..(7): 2′ O-methyl modified nucleoside
  modified_base (8)..(8): 2′ O-methyl modified nucleoside
  modified_base (9)..(9): 2′ O-methyl modified nucleoside
  modified_base (10)..(10): 2′ O-methyl modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ fluoro-modified nucleoside
  modified_base (15)..(15): 2′ O-methyl modified nucleoside
  modified_base (16)..(16): 2′ fluoro-modified nucleoside
  modified_base (17)..(17): 2′ O-methyl modified nucleoside
  modified_base (18)..(18): 2′ O-methyl modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 10 - RNA, 21 nt, Artificial Sequence
  Description of Artificial Sequence Synthetic oligonucleotide
  modified_base (1)..(1): 2′ O-methyl modified nucleoside
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′ O-methyl modified nucleoside
  modified_base (4)..(4): 2′ fluoro-modified nucleoside
  modified_base (5)..(5): 2′ O-methyl modified nucleoside
  modified_base (6)..(6): 2′ fluoro-modified nucleoside
  modified_base (7)..(7): 2′ O-methyl modified nucleoside
  modified_base (8)..(8): 2′ O-methyl modified nucleoside
  modified_base (9)..(9): 2′ O-methyl modified nucleoside
  modified_base (10)..(10): 2′ O-methyl modified nucleoside
  modified_base (11)..(11): 2′ O-methyl modified nucleoside
  modified_base (12)..(12): 2′ O-methyl modified nucleoside
  modified_base (13)..(13): 2′ O-methyl modified nucleoside
  modified_base (14)..(14): 2′ fluoro-modified nucleoside
  modified_base (15)..(15): 2′ O-methyl modified nucleoside
  modified_base (16)..(16): 2′ fluoro-modified nucleoside
  modified_base (17)..(17): 2′ O-methyl modified nucleoside
  modified_base (18)..(18): 2′ O-methyl modified nucleoside
  modified_base (19)..(19): 2′ O-methyl modified nucleoside
  misc_feature (19)..(20): Phosphorothioate linkage
  modified_base (20)..(20): 2′ O-methyl modified nucleoside
  misc_feature (20)..(21): Phosphorothioate linkage
  modified_base (21)..(21): 2′ O-methyl modified nucleoside
nnnnnnnnnnnnnnnnnnnnn

SEQ ID NO: 11 - DNA, 20 bp, Artificial Sequence
  Description of Artificial Sequence Synthetic oligonucleotide
  Description of Combined DNA/RNA Molecule Synthetic oligonucleotide
  modified_base (1)..(1): 2′ O-methyl or 2′O-methoxyethyl-modified nucleoside
  misc_feature (1)..(2): Phosphorothioate linkage
  modified_base (2)..(2): 2′ O-methyl or 2′O-methoxyethyl-modified nucleoside
  misc_feature (2)..(3): Phosphorothioate linkage
  modified_base (3)..(3): 2′ O-methyl or 2′O-methoxyethyl-modified nucleoside
  misc_feature (3)..(4): Phosphorothioate linkage
  modified_base (4)..(4): 2′ O-methyl or 2′O-methoxyethyl-modified nucleoside
  misc_feature (4)..(5): Phosphorothioate linkage
  modified_base (5)..(5): 2′ O-methyl or 2′O-methoxyethyl-modified nucleoside
  misc_feature (5)..(6): Phosphorothioate linkage
  modified_base (6)..(6): Any deoxynucleotide
  misc_feature (6)..(7): Phosphorothioate linkage
  modified_base (7)..(7): Any deoxynucleotide
  misc_feature (7)..(8): Phosphorothioate linkage
  modified_base (8)..(8): Any deoxynucleotide
  misc_feature (8)..(9): Phosphorothioate linkage
  modified_base (9)..(9): Any deoxynucleotide
  misc_feature (9)..(10): Phosphorothioate linkage
  modified_base (10)..(10): Any deoxynucleotide
  misc_feature (10)..(11): Phosphorothioate linkage
  modified_base (11)..(11): Any deoxynucleotide
  misc_feature (11)..(12): Phosphorothioate linkage
  modified_base (12)..(12): Any deoxynucleotide
  misc_feature (12)..(13): Phosphorothioate linkage
  modified_base (13)..(13): Any deoxynucleotide
  misc_feature (13)..(14): Phosphorothioate linkage
  modified_base (14)..(14): Any deoxynucleotide
  misc_feature (14)..(15): Phosphorothioate linkage
  modified_base (15)..(15): Any deoxynucleotide
  misc_feature (15)..(16): Phosphorothioate linkage
  modified_base (16)..(16): 2′ O-methyl or 2′O-methoxyethyl-modified nucleoside
  misc_feature (16)..(17): Phosphorothioate linkage
  modified_base (17)..(17): 2′ O-methyl or 2′O-methoxyethyl-modified nucleoside
  misc_feature (17)..(18): Phosphorothioate linkage
  modified_base (18)..(18): 2′ O-methyl or 2′O-methoxyethyl-modified nucleoside
  misc_feature (18)..(19): Phosphorothioate linkage
  modified_base (19)..(19): 2′ O-methyl or 2′O-methoxyethyl-modified nucleoside
  misc_feature (19)..(20): Phosphorothioate linkage
  modified_base (20)..(20): 2′ O-methyl or 2′O-methoxyethyl-modified nucleoside
nnnnnnnnnnnnnnnnnnnn

SEQ ID NO: 12 - protein, 5 aa, Artificial Sequence
  Description of Artificial Sequence Synthetic peptide
  MOD_RES (4)..(4): D-amino acid
RGDFC

SEQ ID NO: 13 - protein, 5 aa, Artificial Sequence
  Description of Artificial Sequence Synthetic peptide
  MOD_RES (4)..(4): D-amino acid
RGDFK

SEQ ID NO: 14 - protein, 4 aa, Artificial Sequence
  Description of Artificial Sequence Synthetic peptide
  MOD_RES (4)..(4): D-amino acid
RGDF

(19) United States
(12) Patent Application Publication
(10) Pub. No.: US 2023/0407299 A1
(43) Pub. Date: Dec. 21, 2023

(54) TREATMENT OF SOS2 RELATED DISEASES AND DISORDERS

(71) Applicant: Empirico Inc., San Diego, CA (US)

(72) Inventors: Omri GOTTESMAN, San Diego, CA (US); Shannon BRUSE, San Diego, CA (US); Brian CAJES, San Diego, CA (US); David JAKUBOSKY, San Diego, CA (US); Sarah E. KLEINSTEIN, San Diego, CA (US); John VEKICH, San Diego, CA (US)

(21) Appl. No.: 18/037,959

(22) PCT Filed: Nov. 22, 2021
(86) PCT No.: PCT/US2021/060365

§ 371 Date: May 19, 2023

Related U.S. Application Data
(60) Provisional application No. 63/117,862, filed on Nov. 24, 2020.

(51) Int. Cl. C12N 15/113; A61P 13/12; A61P 27/06; A61P 1/16

(52) U.S. Cl. CPC C12N 15/113; A61P 13/12; A61P 27/06; A61P 1/16; C12N 2310/14; C12N 2310/11; C12N 2310/315; C12N 2310/321; C12N 2310/322

(57) ABSTRACT

Disclosed herein are compositions comprising an oligonucleotide that targets SOS Ras/Rho guanine nucleotide exchange factor 2 (SOS2). The oligonucleotide may include a small interfering RNA (siRNA) or an antisense oligonucleotide (ASO). Also provided herein are methods of treating conditions associated with SOS2 mutations that include providing an oligonucleotide that targets SOS2 to a subject.

### CROSS-REFERENCE

This application claims the benefit of U.S. Provisional Application No. 63/117,862, filed Nov. 24, 2020, which application is incorporated herein by reference.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Nov. 17, 2021, is named 54462-725_601_SEtxt and is 33,371 bytes in size.

### BACKGROUND OF THE INVENTION

Indications such as chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss may affect a wide variety of persons. Improved therapeutics are needed.

### SUMMARY OF THE INVENTION

Disclosed herein, in some embodiments, are compositions comprising an oligonucleotide that targets SOS2 and when administered to a subject in an effective amount increases an estimated glomerular filtration rate, or decreases a creatinine, blood urea nitrogen, proteinuria or microalbuminuria measurement. In some embodiments, the estimated glomerular filtration rate is increased, or the creatinine, blood urea nitrogen, proteinuria or microalbuminuria measurement is decreased, by about 10% or more, as compared to prior to administration. Disclosed herein, in some embodiments, are compositions comprising an oligonucleotide that targets SOS2 and when administered to a subject in an effective amount decreases a blood urate measurement. In some embodiments, the blood urate measurement is decreased by about 10% or more, as compared to prior to administration. Disclosed herein, in some embodiments, are compositions comprising an oligonucleotide that targets SOS2 and when administered to a subject in an effective amount decreases a systolic or diastolic blood pressure measurement. In some embodiments, the systolic or diastolic blood pressure measurement is decreased by about 10% or more, as compared to prior to administration. Disclosed herein, in some embodiments, are compositions comprising an oligonucleotide that targets SOS2 and when administered to a subject in an effective amount decreases an intraocular pressure measurement. In some embodiments, the intraocular pressure measurement is decreased by about 10% or more, as compared to prior to administration. Disclosed herein, in some embodiments, are compositions comprising an oligonucleotide that targets SOS2 and when administered to a subject in an effective amount decreases a hemoglobin A1C measurement. In some embodiments, the hemoglobin A1C measurement is decreased by about 10% or more, as compared to prior to administration. Disclosed herein, in some embodiments, are compositions comprising an oligonucleotide that targets SOS2 and when administered to a subject in an effective amount decreases an alanine aminotransferase, aspartate aminotransferase, or liver fat percentage measurement. In some embodiments, the alanine aminotransferase, aspartate aminotransferase, or liver fat percentage measurement is decreased by about 10% or more, as compared to prior to administration. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. Disclosed herein, in some embodiments, are compositions comprising an oligonucleotide that inhibits the expression of SOS2 wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 1. In some embodiments, the oligonucleotide comprises an antisense oligonucleotide (ASO). Disclosed herein, in some embodiments, are compositions comprising an oligonucleotide that inhibits the expression of SOS2 wherein the oligonucleotide comprises an ASO that is complementary to a nucleoside sequence comprising about 12-30 contiguous nucleosides of SEQ ID NO: 1. In some embodiments, the ASO is 12-30 nucleosides in length. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the oligonucleotide comprises 2, 3, 4, 5, 6, 7, 8, 9, 10, 11, 12, 13, 14, 15, 16, 17, 18, 19, 20, or 21 modified nucleosides. In some embodiments, the oligonucleotide comprises an N-acetylgalactosamine (GalNAc) ligand, an arginine-glycine-aspartic acid (RGD) peptide, or a cholesterol ligand. Disclosed herein, in some embodiments, are methods of treating chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss in a subject in need thereof comprising administering to the subject a composition described herein.

### DETAILED DESCRIPTION OF THE INVENTION

Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, kidney, liver, adipocyte, or eye) and a relevant indication.

SOS Ras/Rho guanine nucleotide exchange factor 2 (SOS2) encodes son of sevenless homolog 2 (SOS2), a regulatory protein that may be involved in the positive regulation of ras proteins. SOS2 may map to 14q21 within the human genome. SOS2 may activate RAC1. Mutations in SOS2 may relate to Noonan syndrome. Here it is shown that loss-of-function SOS2 variants resulted in protective associations. Therefore, inhibition of SOS2 may serve as a therapeutic for treatment of SOS2-related diseases and disorders. In particular, it is shown here that loss-of-function genetic variants of 5052 may be protective for chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss (e.g. androgenetic alopecia). Therefore, inhibition of SOS2 may serve as a therapeutic for treatment of these indications.

Disclosed herein are compositions comprising an oligonucleotide that targets SOS2. The oligonucleotide may include a small interfering RNA (siRNA) or an antisense oligonucleotide (ASO). Also provided herein are methods of treating chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss (e.g. androgenetic alopecia) by providing an oligonucleotide that targets SOS2 to a subject in need thereof.

### I. Compositions

Disclosed herein, in some embodiments, are compositions comprising an oligonucleotide. In some embodiments, the composition comprises an oligonucleotide that targets SOS Ras/Rho guanine nucleotide exchange factor 2 (SOS2). In some embodiments, the composition consists of an oligonucleotide that targets SOS2. In some embodiments, the oligonucleotide reduces SOS2 mRNA expression in the subject. In some embodiments, the oligonucleotide reduces son of sevenless homolog 2 (SOS2) protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder as described herein.

Some embodiments include a composition comprising an oligonucleotide that targets SOS2 and when administered to a subject in an effective amount decreases SOS2 mRNA or SOS2 protein levels in a cell, fluid or tissue. In some embodiments, the composition comprises an oligonucleotide that targets SOS2 and when administered to a subject in an effective amount decreases SOS2 mRNA levels in a cell or tissue. In some embodiments, the cell is a liver cell (e.g. hepatocyte), kidney cell (e.g. podocyte), eye cell, or adipocyte. In some embodiments, the tissue is liver, kidney, eye, or adipose tissue. In some embodiments, the SOS2 mRNA levels are decreased by about 2.5% or more, about 5% or more, or about 7.5% or more, as compared to prior to administration. In some embodiments, the SOS2 mRNA levels are decreased by about 10% or more, as compared to prior to administration. In some embodiments, the SOS2 mRNA levels are decreased by about 20% or more, about 30% or more, about 40% or more, about 50% or more, about 60% or more, about 70% or more, about 80% or more, about 90% or more, about 95% or more, or about 100%, as compared to prior to administration. In some embodiments, the v mRNA levels are decreased by no more than about 2.5%, no more than about 5%, or no more than about 7.5%, as compared to prior to administration. In some embodiments, the SOS2 mRNA levels are decreased by no more than about 10%, as compared to prior to administration. In some embodiments, the SOS2 mRNA levels are decreased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the SOS2 mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets SOS2 and when administered to a subject in an effective amount decreases SOS2 protein levels in a cell or tissue. In some embodiments, the cell is a liver cell (e.g. hepatocyte), kidney cell (e.g. podocyte), eye cell, or adipocyte. In some embodiments, the tissue is liver, kidney, eye, or adipose tissue. In some embodiments, the SOS2 protein levels are decreased by about 2.5% or more, about 5% or more, or about 7.5% or more, as compared to prior to administration. In some embodiments, the SOS2 protein levels are decreased by about 10% or more, as compared to prior to administration. In some embodiments, the SOS2 protein levels are decreased by about 20% or more, about 30% or more, about 40% or more, about 50% or more, about 60% or more, about 70% or more, about 80% or more, about 90% or more, or about 100%, as compared to prior to administration. In some embodiments, the SOS2 protein levels are decreased by no more than about 2.5%, no more than about 5%, or no more than about 7.5%, as compared to prior to administration. In some embodiments, the SOS2 protein levels are decreased by no more than about 10%, as compared to prior to administration. In some embodiments, the SOS2 protein levels are decreased by no more than about 20%, no more than about 30%, no more than about 40%, no more than about 50%, no more than about 60%, no more than about 70%, no more than about 80%, no more than about 90%, about 95% or more, or no more than about 100%, as compared to prior to administration. In some embodiments, the SOS2 protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets SOS2 and when administered to a subject in an effective amount decreases a kidney disease-related parameter. In some embodiments, the kidney disease comprises chronic kidney disease (CKD). In some embodiments, the kidney disease comprises diabetic nephropathy. The kidney disease-related parameter may include a blood creatinine measurement. The kidney disease-related parameter may include a blood urea nitrogen (BUN) measurement. The kidney disease-related parameter may include a BUN/creatinine measurement. The parameter may include a proteinuria measurement. The parameter may include a microalbuminuria measurement. In some embodiments, the kidney disease-related parameter is decreased by about 2.5% or more, about 5% or more, or about 7.5% or more, as compared to prior to administration. In some embodiments, the kidney disease-related parameter is decreased by about 10% or more, as compared to prior to administration. In some embodiments, the kidney disease-related parameter is decreased by about 20% or more, about 30% or more, about 40% or more, about 50% or more, about 60% or more, about 70% or more, about 80% or more, or about 90% or more, as compared to prior to administration. In some embodiments, the kidney disease-related parameter is decreased by no more than about 2.5%, no more than about 5%, or no more than about 7.5%, as compared to prior to administration. In some embodiments, the kidney disease-related parameter is decreased by no more than about 10%, as compared to prior to administration. In some embodiments, the kidney disease-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the kidney disease-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount increases a kidney disease-related parameter. The kidney disease-related parameter may include a glomerular filtration rate (GFR). The kidney disease-related parameter may include an estimated glomerular filtration rate (eGFR). In some embodiments, the kidney disease-related parameter is increased by about 2.5% or more, about 5% or more, or about 7.5% or more, as compared to prior to administration. In some embodiments, the kidney disease-related parameter is increased by about 10% or more, as compared to prior to administration. In some embodiments, the kidney disease-related parameter is increased by about 20% or more, about 30% or more, about 40% or more, about 50% or more, about 60% or more, about 70% or more, about 80% or more, about 90% or more, or about 100% or more, as compared to prior to administration. In some embodiments, the kidney disease-related parameter is increased by about 200% or more, about 300% or more, about 400% or more, about 500% or more, about 600% or more, about 700% or more, about 800% or more, about 900% or more, or about 1000% or more, as compared to prior to administration. In some embodiments, the kidney disease-related parameter is increased by no more than about 2.5%, no more than about 5%, or no more than about 7.5%, as compared to prior to administration. In some embodiments, the kidney disease-related parameter is increased by no more than about 10%, as compared to prior to administration. In some embodiments, the kidney disease-related parameter is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the kidney disease-related parameter is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the kidney disease-related parameter is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets SOS2 and when administered to a subject in an effective amount decreases a gout-related or hyperuricemia-related parameter. The gout-related or hyperuricemia-related parameter may include a blood urate measurement. In some embodiments, the gout-related or hyperuricemia-related parameter is decreased by about 2.5% or more, about 5% or more, or about 7.5% or more, as compared to prior to administration. In some embodiments, the gout-related or hyperuricemia-related parameter is decreased by about 10% or more, as compared to prior to administration. In some embodiments, the gout-related or hyperuricemia-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the gout-related or hyperuricemia-related parameter is decreased by no more than about 2.5%, no more than about 5%, or no more than about 7.5%, as compared to prior to administration. In some embodiments, the gout-related or hyperuricemia-related parameter is decreased by no more than about 10%, as compared to prior to administration. In some embodiments, the gout-related or hyperuricemia-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the gout-related or hyperuricemia-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a cerebrovascular disease-related parameter. The cerebrovascular disease-related parameter may include a hypertension-related parameter. The hypertension-related parameter may include a systolic blood pressure measurement. The hypertension-related parameter may include a diastolic blood pressure measurement. In some embodiments, the hypertension-related parameter is decreased by about 2.5% or more, about 5% or more, or about 7.5% or more, as compared to prior to administration. In some embodiments, the hypertension-related parameter is decreased by about 10% or more, as compared to prior to administration. In some embodiments, the hypertension-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the hypertension-related parameter is decreased by no more than about 2.5%, no more than about 5%, or no more than about 7.5%, as compared to prior to administration. In some embodiments, the hypertension-related parameter is decreased by no more than about 10%, as compared to prior to administration. In some embodiments, the hypertension-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the hypertension-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a glaucoma-related parameter. The glaucoma-related parameter may include a intraocular pressure measurement. In some embodiments, the glaucoma-related parameter is decreased by about 2.5% or more, about 5% or more, or about 7.5% or more, as compared to prior to administration. In some embodiments, the glaucoma-related parameter is decreased by about 10% or more, as compared to prior to administration. In some embodiments, the glaucoma-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the glaucoma-related parameter is decreased by no more than about 2.5%, no more than about 5%, or no more than about 7.5%, as compared to prior to administration. In some embodiments, the glaucoma-related parameter is decreased by no more than about 10%, as compared to prior to administration. In some embodiments, the glaucoma-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the glaucoma-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a metabolic disorder-related parameter. In some embodiments, the metabolic disorder comprises obesity. In some embodiments, the metabolic disorder comprises metabolic syndrome. In some embodiments, the metabolic disorder comprises diabetes. In some embodiments, the diabetes comprises type II diabetes. The metabolic disorder-related parameter may include a hemoglobin A1C measurement. In some embodiments, the metabolic disorder-related parameter is decreased by about 2.5% or more, about 5% or more, or about 7.5% or more, as compared to prior to administration. In some embodiments, the metabolic disorder-related parameter is decreased by about 10% or more, as compared to prior to administration. In some embodiments, the metabolic disorder-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the metabolic disorder-related parameter is decreased by no more than about 2.5%, no more than about 5%, or no more than about 7.5%, as compared to prior to administration. In some embodiments, the metabolic disorder-related parameter is decreased by no more than about 10%, as compared to prior to administration. In some embodiments, the metabolic disorder-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the metabolic disorder-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a liver disease-related parameter. In some embodiments, the liver disease comprises fibrotic liver disease. In some embodiments, the liver disease comprises non-alcoholic fatty liver disease (NAFLD). The liver disease-related parameter may include an aspartate aminotransferase (AST) measurement. The liver disease-related parameter may include an alanine aminotransferase (ALT) measurement. The liver disease-related parameter may include an AST/ALT ratio. The liver disease-related parameter may include a liver fat percentage measurement. In some embodiments, the liver disease-related parameter is decreased by about 2.5% or more, about 5% or more, or about 7.5% or more, as compared to prior to administration. In some embodiments, the liver disease-related parameter is decreased by about 10% or more, as compared to prior to administration. In some embodiments, the liver disease-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the liver disease-related parameter is decreased by no more than about 2.5%, no more than about 5%, or no more than about 7.5%, as compared to prior to administration. In some embodiments, the liver disease-related parameter is decreased by no more than about 10%, as compared to prior to administration. In some embodiments, the liver disease-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the liver disease-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a hair loss-related parameter. In some embodiments, the hair loss comprises androgenetic alopecia. The hair loss-related parameter may include a hair count measurement. The hair loss-related parameter may include a hair thickness measurement. The hair loss-related parameter may include a hair density measurement. In some embodiments, the hair loss-related parameter is decreased by about 2.5% or more, about 5% or more, or about 7.5% or more, as compared to prior to administration. In some embodiments, the hair loss-related parameter is decreased by about 10% or more, as compared to prior to administration. In some embodiments, the hair loss-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the hair loss-related parameter is decreased by no more than about 2.5%, no more than about 5%, or no more than about 7.5%, as compared to prior to administration. In some embodiments, the hair loss-related parameter is decreased by no more than about 10%, as compared to prior to administration. In some embodiments, the hair loss-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the hair loss-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

### A. siRNAs

In some embodiments, the composition comprises an oligonucleotide that targets SOS Ras/Rho guanine nucleotide exchange factor 2 (SOS2), wherein the oligonucleotide comprises a small interfering RNA (siRNA). In some embodiments, the composition comprises an oligonucleotide that targets SOS2, wherein the oligonucleotide comprises a small interfering RNA (siRNA) comprising a sense strand and an antisense strand.

In some embodiments, the composition comprises an oligonucleotide that inhibits the expression of SOS2, wherein the oligonucleotide comprises an siRNA comprising a sense strand and an antisense strand, wherein the sense strand is 14-30 nucleosides in length. In some embodiments, the composition comprises a sense strange that is at least about 10, 11, 12, 13, 14, 16, 17, 18, 19, 20, 21, 22, 23, 24, 25, 26, 27, 28, 29, or 30 nucleosides in length, or a range defined by any of the two aforementioned numbers. In some embodiments, the composition comprises an antisense strand is 14-30 nucleosides in length. In some embodiments, the composition comprises an antisense strange that is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers.

In some embodiments, the composition comprises an oligonucleotide that inhibits the expression of SOS2, wherein the oligonucleotide comprises an siRNA comprising a sense strand and an antisense strand, each strand is independently about 14-30 nucleosides in length, and at least one of the sense strand and the antisense strand comprises a nucleoside sequence comprising about 14-30 contiguous nucleosides of a full-length human SOS2 mRNA sequence such as SEQ ID NO: 1. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 1.

In some embodiments, the composition comprises an oligonucleotide that inhibits the expression of SOS2, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of SOS2, wherein the oligonucleotide comprises an siRNA comprising a sense strand and an antisense strand, wherein the siRNA binds with a 19mer in a human SOS2 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human SOS2 mRNA.

In some embodiments, the composition comprises an oligonucleotide that inhibits the expression of SOS2, wherein the oligonucleotide comprises an siRNA comprising a sense strand and an antisense strand, wherein the siRNA binds with a 17mer in a non-human primate SOS2 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate SOS2 mRNA.

In some embodiments, the composition comprises an oligonucleotide that inhibits the expression of SOS2, wherein the oligonucleotide comprises an siRNA comprising a sense strand and an antisense strand, wherein the siRNA binds with a 19mer in a human SOS2 mRNA, or a combination thereof. In some embodiments, the siRNA binds with a 12mer, a 13mer, a 14mer, a 15mer, a 16mer, a 17mer, and 18mer, a 19mer, a 20mer, a 21mer, a 22mer, a 23mer, a 24mer, or a 25mer in a human SOS2 mRNA.

In some embodiments, the composition comprises an oligonucleotide that inhibits the expression of SOS2, wherein the oligonucleotide comprises an siRNA comprising a sense strand and an antisense strand, wherein the siRNA binds with a human SOS2 mRNA and less than or equal to 20 human off-targets, with no more than 2 mismatches in the antisense strand. In some embodiments, the siRNA binds with a human SOS2 mRNA and less than or equal to 10 human off-targets, with no more than 2 mismatches in the antisense strand. In some embodiments, the siRNA binds with a human SOS2 mRNA and less than or equal to 30 human off-targets, with no more than 2 mismatches in the antisense strand. In some embodiments, the siRNA binds with a human SOS2 mRNA and less than or equal to 40 human off-targets, with no more than 2 mismatches in the antisense strand. In some embodiments, the siRNA binds with a human SOS2 mRNA and less than or equal to 50 human off-targets, with no more than 2 mismatches in the antisense strand. In some embodiments, the siRNA binds with a human SOS2 mRNA and less than or equal to 10 human off-targets, with no more than 3 mismatches in the antisense strand. In some embodiments, the siRNA binds with a human SOS2 mRNA and less than or equal to 20 human off-targets, with no more than 3 mismatches in the antisense strand. In some embodiments, the siRNA binds with a human SOS2 mRNA and less than or equal to 30 human off-targets, with no more than 3 mismatches in the antisense strand. In some embodiments, the siRNA binds with a human SOS2 mRNA and less than or equal to 40 human off-targets, with no more than 3 mismatches in the antisense strand. In some embodiments, the siRNA binds with a human SOS2 mRNA and less than or equal to 50 human off-targets, with no more than 3 mismatches in the antisense strand.

In some embodiments, the composition comprises an oligonucleotide that inhibits the expression of SOS2, wherein the oligonucleotide comprises an siRNA comprising a sense strand and an antisense strand, siRNA binds with a human SOS2 mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

### B. ASOs

In some embodiments, the composition comprises an oligonucleotide that inhibits the expression of SOS2, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-nucleosides in length. In some embodiments, the ASO is 20 nucleosides in length.

In some embodiments, the composition comprises an oligonucleotide that inhibits the expression of SOS2, wherein the oligonucleotide comprises an ASO about 12-30 nucleosides in length and comprising a nucleoside sequence complementary to about 12-30 contiguous nucleosides of a full-length human SOS2 mRNA sequence such as SEQ ID NO: 1; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 1.

### C. Modification Patterns

In some embodiments, the composition comprises an oligonucleotide that inhibits the expression of SOS2, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. Benefits of the modified internucleoside linkage may include decreased toxicity or improved pharmacokinetics.

In some embodiments, the composition comprises an oligonucleotide that inhibits the expression of SOS2, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of SOS2, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

In some embodiments, the composition comprises an oligonucleotide that inhibits the expression of SOS2, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

In some embodiments, the composition comprises an oligonucleotide that inhibits the expression of SOS2, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

In some embodiments, the composition comprises an oligonucleotide that inhibits the expression of SOS2, wherein the oligonucleotide comprises an arginine-glycine-aspartic acid (RGD) peptide. In some embodiments, the composition comprises an RGD peptide. In some embodiments, the composition comprises an RGD peptide derivative. In some embodiments, the RGD peptide is attached at a 3′ terminus of the oligonucleotide. In some embodiments, the RGD peptide is attached at a 5′ terminus of the oligonucleotide. In some embodiments, the composition comprises a sense strand, and the RGD peptide is attached to the sense strand (e.g. attached to a 5′ end of the sense strand, or attached to a 3′ end of the sense strand). In some embodiments, the composition comprises an antisense strand, and the RGD peptide is attached to the antisense strand (e.g. attached to a 5′ end of the antisense strand, or attached to a 3′ end of the antisense strand). In some embodiments, the composition comprises an RGD peptide attached at a 3′ or 5′ terminus of the oligonucleotide. In some embodiments, the oligonucleotide comprises an RGD peptide and a lipid attached at a 3′ or 5′ terminus of the oligonucleotide. In some embodiments, the RGD peptide comprises Cyclo(-Arg-Gly-Asp-D-Phe-Cys) (SEQ ID NO: 12). In some embodiments, the RGD peptide comprises Cyclo(-Arg-Gly-Asp-D-Phe-Lys) (SEQ ID NO: 13). In some embodiments, the RGD peptide comprises Cyclo(-Arg-Gly-Asp-D-Phe-azido) (SEQ ID NO: 14). In some embodiments, the RGD peptide comprises an amino benzoic acid derived RGD. In some embodiments, the RGD peptide comprises Cyclo(-Arg-Gly-Asp-D-Phe-Cys) (SEQ ID NO: 12), Cyclo(-Arg-Gly-Asp-D-Phe-Lys) (SEQ ID NO: 13), Cyclo(-Arg-Gly-Asp-D-Phe-azido) (SEQ ID NO: 14), an amino benzoic acid derived RGD, or a combination thereof. In some embodiments, the RGD peptide comprises multiple of such RGD peptides. For example, the RGD peptide may include 2, 3, or 4 RGD peptides.

In some embodiments, the composition comprises an oligonucleotide that inhibits the expression of SOS2, wherein the oligonucleotide comprises a cholesterol ligand. In some embodiments, the composition comprises cholesterol. In some embodiments, the composition comprises a cholesterol derivative. In some embodiments, the cholesterol ligand is attached at a 3′ terminus of the oligonucleotide. In some embodiments, the cholesterol ligand is attached at a 5′ terminus of the oligonucleotide. In some embodiments, the composition comprises a sense strand, and the cholesterol ligand is attached to the sense strand (e.g. attached to a 5′ end of the sense strand, or attached to a 3′ end of the sense strand). In some embodiments, the composition comprises an antisense strand, and the cholesterol ligand is attached to the antisense strand (e.g. attached to a 5′ end of the antisense strand, or attached to a 3′ end of the antisense strand). In some embodiments, the composition comprises a cholesterol ligand attached at a 3′ or 5′ terminus of the oligonucleotide.

1. siRNA Modification Patterns

In some embodiments, the composition comprises an oligonucleotide that inhibits the expression of SOS2 wherein the oligonucleotide comprises an siRNA comprising a sense strand and an antisense strand, wherein the sense strand comprises modification pattern 1S: (SEQ ID NO: 2), wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′ (SEQ ID NO: 3), wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′ (SEQ ID NO: 4), wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-Lipid-3′ (SEQ ID NO: 5), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-Lipid-3′ (SEQ ID NO: 6), wherein “Nf” is a 2′ fluoro-modified nucleoside, “n” is a 2′ O-methyl modified nucleoside, “s” is a phosphorothioate linkage, and N comprises one or more nucleosides.

In some embodiments, the composition comprises an oligonucleotide that inhibits the expression of SOS2 wherein the oligonucleotide comprises an siRNA comprising a sense strand and an antisense strand, wherein the antisense strand comprises modification pattern 1AS: (SEQ ID NO: 7), wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: (SEQ ID NO: 8), wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: (SEQ ID NO: 9), wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: (SEQ ID NO: 10), wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of SOS2 wherein the oligonucleotide comprises an siRNA comprising a sense strand and an antisense strand, wherein the sense strand comprises pattern 15 and the antisense strand comprises pattern 1AS, 2AS, 3AS, or 4AS. In some embodiments, the sense strand comprises pattern 2S and the antisense strand comprises pattern 1AS, 2AS, 3AS, or 4AS. In some embodiments, the sense strand comprises pattern 3S and the antisense strand comprises pattern 1AS, 2AS, 3AS, or 4AS. In some embodiments, the sense strand comprises pattern 4S and the antisense strand comprises pattern 1AS, 2AS, 3AS, or 4AS. In some embodiments, the sense strand comprises modification pattern 1AS, 2AS, 3AS, or 4AS. In some embodiments, the antisense strand comprises modification pattern 1S, 2S, 3S, 4S, or 5S. In some embodiments, the sense strand or the antisense strand comprises modification pattern ASO1.

2. ASO Modification Patterns

In some embodiments, the composition comprises an oligonucleotide that inhibits the expression of SOS2, wherein the oligonucleotide comprises an antisense oligonucleotide (ASO). In some embodiments, the ASO comprises modification pattern ASO1: (SEQ ID NO: 11), wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S, 2S, 3S, 4S, 5S, 1AS, 2AS, 3AS, or 4AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### II. Methods and Uses

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

### A. Disorders

Some embodiments of the methods described herein include treating a disorder in a subject in need thereof. Some embodiments include administering a composition described herein to a subject having the disorder. In some embodiments, the disorder is a chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss disorder. In some embodiments, the disorder comprises a kidney disease. In some embodiments, the disorder comprises chronic kidney disease. In some embodiments, the disorder comprises diabetic nephropathy. In some embodiments, the disorder comprises gout. In some embodiments, the disorder comprises hyperuricemia. In some embodiments, the disorder comprises hypertension. In some embodiments, the disorder comprises cerebrovascular disease. In some embodiments, the disorder comprises a metabolic disorder. In some embodiments, the disorder comprises diabetes. In some embodiments, the disorder comprises type 2 diabetes. In some embodiments, the disorder comprises metabolic syndrome. In some embodiments, the disorder comprises obesity. In some embodiments, the disorder comprises glaucoma. In some embodiments, the disorder comprises a liver disease. In some embodiments, the disorder comprises non-alcoholic fatty liver disease. In some embodiments, the disorder comprises fibrotic liver disease. In some embodiments, the disorder comprises hair loss.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female.

In some embodiments, the subject has a body mass index (BMI) of 25, 26, 27, 28, 29, 30, 31, 32, 33, 34, 35, 36, 37, 38, 39, 40, 41, 42, 43, 44, 45, 46, 47, 48, 49, 50, or more, or a range defined by any two of the aforementioned integers. In some embodiments, the subject is overweight. In some embodiments, the subject has a BMI of 25 or more. In some embodiments, the subject has a BMI of 25-29. In some embodiments, the subject is obese. In some embodiments, the subject has a BMI of 30 or more. In some embodiments, the subject has a BMI of 30-39. In some embodiments, the subject has a BMI of 40-50. In some embodiments, the subject has a BMI of 25-50.

In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

### C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline glomerular filtration rate (GFR) or estimated glomerular filtration rate (eGFR) measurement, a baseline creatinine measurement, a baseline blood urea nitrogen (BUN) measurement, a baseline proteinuria measurement, a baseline microalbuminuria measurement, a baseline blood urate measurement, a baseline systolic blood pressure (SBP) measurement, a baseline diastolic blood pressure (DBP) measurement, a baseline intraocular pressure (IOP) measurement, a baseline hemoglobin A1C measurement, a baseline alanine aminotransferase (ALT) measurement, a baseline aspartate aminotransferase (AST) measurement, a baseline liver fat percentage (LFP) measurement, a baseline liver fibrosis measurement, a baseline hair count measurement, a baseline hair thickness measurement, a baseline hair density measurement, a baseline SOS2 protein measurement, or a baseline SOS2 mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device. In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline GFR or eGFR measurement. In some embodiments, the baseline measurement is a baseline GFR measurement. In some embodiments, the baseline measurement is a baseline eGFR measurement. The baseline GFR or eGFR measurement may be indicated in units of volume per time (e.g. mL/min). The baseline GFR measurement may be obtained using a baseline clearance measurement such as a baseline creatinine clearance measurement. The baseline GFR may also be determined by injecting insulin, sinistrin, a radioactive tracer, or cystatin C, and determining a baseline clearance rate. The baseline eGFR measurement may be also be obtained using a clearance estimate such as an estimation of serum creatinine clearance. The baseline GFR or eGFR may be 100-130 mL/min/1.73 m², 90-100 mL/min/1.73 m². The baseline GFR or eGFR may be below or 100 mL/min/1.73 m 2. The baseline GFR or eGFR may be indicative of normal kidney function, CKD1, CKD2, CKD3, CKD4, or CKD5, as indicated by the following kidney function index:
Normal kidney function—GFR above 90 mL/min/1.73 m² (optionally with no proteinuria)
CKD1—GFR above 90 mL/min/1.73 m² (optionally with evidence of kidney damage)
CKD2 (mild)—GFR of 60 to 89 mL/min/1.73 m² (optionally with evidence of kidney damage)
CKD3 (moderate)—GFR of 30 to 59 mL/min/1.73 m²
CKD4 (severe)—GFR of 15 to 29 mL/min/1.73 m²
CKD5 kidney failure—GFR less than 15 mL/min/1.73 m²

In some embodiments, the baseline measurement is a baseline creatinine measurement. In some embodiments, the baseline creatinine measurement is a baseline creatinine concentration. In some embodiments, the baseline creatinine measurement is a baseline circulating (e.g. serum or plasma) creatinine measurement. In some embodiments, the baseline creatinine measurement is a baseline urine creatinine measurement. In some embodiments, the baseline creatinine measurement is obtained by an assay such as an immunoassay, a colorimetric assay, or a fluorescence assay. The baseline circulating creatinine measurement may be about mg/dL. The baseline creatinine may be measured in a fluid sample. The baseline circulating creatinine measurement may be above 1.3 mg/dL. The baseline circulating creatinine measurement may be within, above, or below a reference range. The baseline urine creatinine measurement may be within, above, or below a reference range. Typical human reference ranges for serum creatinine are 0.5 mg/dL to 1.0 mg/dL for women or 0.7 mg/dL to 1.2 mg/dL for men. The significance of a single creatinine value may be interpreted in light of the patient's muscle mass. A patient with a greater muscle mass may have a higher creatinine concentration. While a baseline serum creatinine of 2.0 mg/dL (177 μmol/L) may indicate normal kidney function in a male body builder, a serum creatinine of 1.6 mg/dL (110 μmol/L) may indicate significant renal disease in an elderly female. Males may typically produce approximately 150 μmol to 200 μmol of creatinine per kilogram of body weight per 24 h while females may produce approximately 100 μmol/kg/24 h to 150 μmol/kg/24 h. In normal circumstances, all this daily creatinine production is excreted in the urine, which may be included in a baseline urine creatinine measurement.

In some embodiments, the baseline measurement is a baseline blood urea nitrogen (BUN) measurement. In some embodiments, the baseline BUN measurement is a baseline BUN concentration. In some embodiments, the baseline BUN measurement is a baseline circulating BUN measurement. In some embodiments, the baseline BUN measurement is obtained by an assay such as an immunoassay, a colorimetric assay, or a fluorescence assay. In some embodiments, the baseline BUN is 6-20 mg/dL. In some embodiments, the baseline BUN is over 20 mg/dL. A normal BUN range is 6-20 mg/dL. In some embodiments, the baseline measurement is a baseline BUN/creatinine ratio.

In some embodiments, the baseline measurement is a baseline proteinuria measurement. “Proteinuria” may describe an increase (e.g. a moderate increase) in a level of urine protein. The baseline proteinuria measurement may be indicated as a concentration, a ratio, or a mass/unit time (e.g. mg/mmol urine, protein/creatinine, or mg protein/hr). In some embodiments, the baseline proteinuria measurement includes a baseline proteinuria concentration. In some embodiments, the baseline proteinuria measurement is a baseline urine protein measurement. In some embodiments, the baseline proteinuria measurement is obtained by an assay such as an immunoassay, a colorimetric assay, or a fluorescence assay. In some embodiments, the baseline proteinuria measurement is indicative of proteinuria in the subject. Proteinuria can be diagnosed from a 24-hour urine collection or, from an elevated concentration in a spot sample. In some embodiments, the baseline measurement is a baseline urine protein/creatinine ratio.

In some embodiments, the baseline measurement is a baseline microalbuminuria measurement. “Microalbuminuria” may describe an increase (e.g. a moderate increase) in a level of urine albumin. The baseline microalbuminuria measurement may be indicated as a concentration, a ratio, or a mass/unit time (e.g. mg/mmol urine, albumin/creatinine, or mg albumin/hr). In some embodiments, the baseline microalbuminuria measurement includes a baseline microalbuminuria concentration. In some embodiments, the baseline microalbuminuria measurement is a baseline urine microalbuminuria measurement. In some embodiments, the baseline microalbuminuria measurement is obtained by an assay such as an immunoassay, a colorimetric assay, or a fluorescence assay. In some embodiments, the baseline microalbuminuria measurement is indicative of microalbuminuria in the subject. Microalbuminuria can be diagnosed from a 24-hour urine collection (between 30-300 mg/24 hours) or, from an elevated concentration in a spot sample (20 to 200 mg/1). In some embodiments, the baseline measurement is a baseline urine albumin/creatinine ratio. The baseline microalbuminuria measurement may include a microalbuminuria measurement within a range or amount defined in Table 1.

TABLE 1
Microalbuminuria reference values

| | | Lower | Upper | |
|---|---|---|---|---|
| | Individual | limit | limit | Unit |
| 24 h urine | | 30 | 300 | mg/24 h (milligram |
| collection | | | | albumin per 24 hours) |
| Short-time | | 20 | 200 | μg/min (microgram |
| urine collection | | | | albumin per minute) |
| Spot urine | | 30 | 300 | mg/L (milligram |
| albumin sample | | | | albumin per liter |
| | | | | of urine) |
| Spot urine | Women | 3.5 | 25 or 35 | mg/mmol (milligram |
| albumin/ | | | | albumin per millimole |
| creatinine | | | | creatinine) |
| ratio | | 30 | 400 | μg/mg (microgram |
| | | | | albumin per milligram |
| | | | | creatinine) |
| | Men | 2.5 or 3.5 | 25 or 35 | mg/mmol |
| | | 30 | 300 | μg/mg |

In some embodiments, the baseline measurement is a baseline blood urate measurement. In some embodiments, the baseline blood urate measurement is a baseline blood urate concentration. In some embodiments, the baseline blood urate measurement is obtained by an assay such as an immunoassay, a colorimetric assay, or a fluorescence assay. In some embodiments, the baseline blood urate measurement is indicative of hyperuricemia. Serum uric acid concentrations greater than 6 mg/dL (e.g. for females), greater than 7 mg/dL (e.g. for men), or greater than 5.5 mg/dL (e.g. for a subject under 18 years old) may be indicative of hyperuricemia.

Some embodiments of the methods described herein include obtaining the baseline measurement of the subject by measuring blood pressure (e.g. systolic or diastolic) with a sphygmomanometer in which a healthcare professional places a cuff around an arm of the subject and inflates the cuff with a pump until the circulation is cut off. A small valve slowly deflates the cuff, and the healthcare professional measures the pressure with the aid of a stethoscope that is placed over the arm of the subject in order to listen for the sound of the blood pulsing through the arteries. The first measurement in which blood rushes is the systolic blood pressure (SBP), and after the sound fades, the second number indicates the diastolic blood pressure (DBP), which is a measure the blood pressure of the heart at rest. The mean arterial pressure (MAP) is an average blood pressure of the subject during a single cardiac cycle. The MAP can be measured directly using methods such as applanation tonometry or it can be approximated by using a formula in which the diastolic blood pressure is doubled and added to the systolic blood pressure and that composite sum is then divided by 3 to estimate MAP.

In some embodiments, the baseline measurement is a baseline systolic blood (SBP) pressure measurement. In some embodiments, the baseline SBP measurement is measured in mm of mercury (mm Hg). In some embodiments, the SBP measurement is obtained with a sphygmomanometer. The baseline SBP measurement may be indicative of normal blood pressure. For most adults, normal SBP at rest is within the range of 100-130 mmHg. For most adults, hypertension is present if the resting blood pressure is persistently at or above 130/80 or 140/90 mmHg. The baseline SBP measurement may be indicative of hypertension (e.g. at least 130 mmHg, or at least 140 mmHg). The baseline SBP measurement may include a baseline cerebral SBP measurement.

In some embodiments, the baseline measurement is a baseline diastolic blood (DBP) pressure measurement. In some embodiments, the baseline DBP measurement is measured in mm Hg. In some embodiments, the DBP measurement is obtained with a sphygmomanometer. The baseline DBP measurement may be indicative of normal blood pressure. For most adults, normal DBP at rest is within the range of 60-80 mmHg. The baseline DBP measurement may be indicative of hypertension (e.g. at least 80 mmHg, or at least 90 mmHg). The baseline DBP measurement may include a baseline cerebral DBP measurement.

In some embodiments, the baseline measurement is a baseline intraocular pressure (TOP) measurement. The baseline IOP may be measured using a tonometer. The baseline IOP measurement may be in millimeters of mercury (mmHg). The baseline IOP measurement may be indicative of a normal IOP. The baseline IOP measurement may be indicative of abnormal or high IOP. A normal IPO measurement may be between 10 mmHg and 20 mmHg. The baseline IOP measurement may be above 20 mmHg.

In some embodiments, the baseline measurement is a baseline hemoglobin A1C measurement. In some embodiments, the baseline hemoglobin A1C measurement is a baseline hemoglobin A1C concentration. In some embodiments, the baseline hemoglobin A1C measurement is a baseline circulating hemoglobin A1C measurement. In some embodiments, the baseline hemoglobin A1C measurement is obtained by an assay such as an immunoassay, a colorimetric assay, a fluorescence assay, or HPLC. The baseline hemoglobin A1C measurement may be indicative of a healthy normal A1C measurement. The healthy normal hemoglobin A1C measurement may be below 48 mmol/mol (6.5 DCCT %). The healthy normal hemoglobin A1C measurement may be below 53 mmol/mol (7.0 DCCT %). The baseline hemoglobin A1C measurement may be indicative of diabetes of pre-diabetes. A baseline hemoglobin A1C measurement above 48 mmol/mol, or above 53 mmol/mol may indicate diabetes of pre-diabetes. The baseline hemoglobin A1C measurement may be indicative of diabetes. The baseline hemoglobin A1C measurement may be indicative of pre-diabetes. In some cases, the baseline hemoglobin A1C measurement is below 5.7 DCCT % (e.g. indicative of a normal healthy diagnosis). In some cases, the baseline hemoglobin A1C measurement is between 5.7 and 6.4 DCCT % (e.g. indicative of prediabetes). In some cases, the baseline hemoglobin A1C measurement is above 6.4 DCCT % (e.g. indicative of diabetes).

In some embodiments, the baseline measurement is a baseline liver enzyme measurement. In some embodiments, the baseline liver enzyme measurement is a baseline alanine aminotransferase (ALT) measurement. In some embodiments, the baseline liver enzyme measurement is a baseline aspartate aminotransferase (AST) measurement. In some embodiments, the baseline liver enzyme measurement comprises an ALT/AST ratio, or comprises an AST/ALT ratio. In some embodiments, the baseline liver enzyme measurement is obtained by an assay such as an immunoassay, a colorimetric assay, a fluorescence assay, or HPLC.

In some embodiments, the baseline measurement is a baseline alanine aminotransferase (ALT) measurement. In some embodiments, the baseline ALT measurement is a baseline ALT concentration (for example, Units/dL). In some embodiments, the baseline ALT measurement is a baseline circulating ALT measurement, for example, a baseline blood, serum, or plasma ALT measurement. In some embodiments, the baseline ALT measurement is obtained by an assay such as an immunoassay, a colorimetric assay, or a fluorescence assay. In some embodiments, the baseline ALT measurement is within a reference range of 34 IU/L or lower (e.g. for a female subject) or within a reference range of 45 IU/L or lower (e.g. for a male subject). In some embodiments, the baseline ALT measurement is above the reference range.

In some embodiments, the baseline measurement is a baseline aspartate aminotransferase (AST) measurement. In some embodiments, the baseline AST measurement is a baseline AST concentration (for example, Units/L). In some embodiments, the baseline AST measurement is a baseline circulating AST measurement, for example, a baseline blood, serum, or plasma AST measurement. In some embodiments, the baseline AST measurement is obtained by an assay such as an immunoassay, a colorimetric assay, or a fluorescence assay. In some embodiments, the baseline AST measurement is within a reference range of 6-34 IU/L (e.g. for a female subject) or within a reference range of 8-40 IU/L (e.g. for a male subject). In some embodiments, the baseline AST measurement is above the reference range. In some embodiments, the baseline AST measurement is below the reference range.

In some embodiments, the baseline measurement is a baseline liver steatosis measurement. In some embodiments, the baseline liver steatosis measurement is a baseline liver fat percentage (LFP) measurement. In some embodiments, the baseline measurement is a baseline LFP measurement. In some embodiments, the baseline LFP measurement is indicated as a mass/mass percentage of fat/total tissue. In some embodiments, the baseline LFP measurement is indicated as a mass/volume percentage of fat/total tissue. In some embodiments, the baseline LFP measurement is indicated as a volume/mass percentage of fat/total tissue. In some embodiments, the baseline LFP measurement is indicated as a volume/volume percentage of fat/total tissue. In some embodiments, the baseline LFP measurement is indicated as a score.

In some embodiments, the baseline LFP measurement is obtained noninvasively. In some embodiments, the baseline LFP measurement is obtained by a medical imaging device. In some embodiments, the baseline LFP measurement is obtained by a device such as a medical resonance imaging (MRI) device, a magnetic resonance spectroscopy device, a computed tomography device, a controlled attenuation parameter (CAP), a transient elastography device, or an ultrasound device. In some embodiments, the baseline LFP measurement is obtained in a liver sample. In some embodiments, the baseline LFP measurement comprises a baseline liver triglyceride measurement. In some embodiments, the baseline LFP measurement is obtained by an assay such as an immunoassay, a colorimetric assay, or a fluorescence assay. In some embodiments, the baseline LFP measurement or the baseline LFP measurement is obtained using a scoring system upon a visual inspection of a sample such as a histological sample. In some embodiments, the baseline LFP measurement or the baseline LFP measurement is obtained using a stain with an affinity to fats, such as a lysochrome diazo dye.

In some embodiments, the baseline measurement is a baseline liver fibrosis measurement. In some embodiments, the baseline liver fibrosis measurement is a baseline liver fibrosis score (LFS). In some embodiments, the LFS comprises a score of 0, 1, 2, 3, or 4, or a range of scores defined by any two of the aforementioned numbers. In some embodiments, the LFS comprises a score of 0-4. In some embodiments, the LFS is obtained using a scoring system exemplified in Table 2. In some embodiments, the baseline LFS measurement is obtained noninvasively. In some embodiments, the baseline LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the baseline LFS measurement is obtained in a liver sample. In some embodiments, the baseline LFS is obtained by an assay such as an immunoassay, a colorimetric assay, or a fluorescence assay. In some embodiments, the LFS is obtained using one or more indirect markers or measures of liver fibrosis such as an aspartate aminotransferase-to-platelet ratio index (APRI), a Fibrosis-4 (FIB-4) index, a Fibrolndex, a Forns Index, a Hepascore, or a FibroTest. In some embodiments, the LFS is obtained using one or more indirect markers or measures of liver fibrosis such as a FIBROSpect test or a FIBROSpect II test. In some embodiments, the baseline LFS is obtained by RT-qPCR or RNA sequencing of one or more fibrosis-related genes such as a collagen gene. In some embodiments, the baseline LFS or the baseline LFS is obtained using a scoring system upon a visual inspection of a sample such as a histological sample. In some embodiments, the baseline LFS or the baseline LFS is obtained using a stain with an affinity to collagen.

TABLE 2
Non-Limiting Examples of Liver Fibrosis Scoring Systems

| Score | IASL | Batts-Ludwig | Metavir |
|---|---|---|---|
| 0 | No fibrosis | No fibrosis | No fibrosis |
| 1 | Mild | Fibrous portal | Periportal fibrotic |
| | fibrosis | expansion | expansion |
| 2 | Moderate | Rare bridges | Periportal septae |
| | fibrosis | or septae | (>1 septum) |
| 3 | Severe | Numerous bridges | Portal-central |
| | fibrosis | or septae | septae |
| 4 | Cirrhosis | Cirrhosis | Cirrhosis |

In some embodiments, the baseline measurement is a baseline hair count. In some embodiments, the baseline hair count is a baseline total hair count. The baseline total hair count may include a baseline vellus hair count and a baseline non-vellus hair count. In some embodiments, the baseline hair count is a baseline vellus hair count. In some embodiments, the baseline hair count is a baseline non-vellus hair count. In some embodiments, the baseline hair count is determined in an area of skin. In some embodiments, the baseline hair count is normalized based on the area of skin. In some embodiments, the baseline hair count is assessed using photography. In some embodiments, the baseline hair count is assessed by phototrichogram. In some embodiments, the baseline hair count is assessed by a macrophotography analysis.

In some embodiments, the baseline measurement is a baseline hair thickness measurement. In some embodiments, the baseline hair thickness measurement is determined in an area of skin. In some embodiments, the baseline hair thickness measurement comprises a width of an individual hair. In some embodiments, the baseline hair thickness measurement comprises widths of multiple individual hairs. In some embodiments, the baseline hair thickness measurement comprises an average of the widths of the multiple individual hairs. In some embodiments, the baseline hair thickness measurement comprises a median of the widths of the multiple individual hairs. The baseline hair thickness measurement may include a baseline vellus hair thickness measurement. The baseline hair thickness measurement may include a baseline non-vellus hair thickness measurement. In some embodiments, the baseline hair thickness measurement is assessed using photography. In some embodiments, the baseline hair thickness measurement is assessed by phototrichogram. In some embodiments, the baseline hair thickness measurement is assessed by a macrophotography analysis.

In some embodiments, the baseline measurement is a baseline hair density measurement. In some embodiments, the baseline hair density measurement is determined in an area of skin. In some embodiments, the baseline hair density measurement comprises a number of hair in the area of skin. In some embodiments, the baseline hair density measurement comprises the number of hair in the area of skin divided by the area of skin. The baseline hair density measurement may include a baseline vellus hair density measurement. The baseline hair density measurement may include a baseline non-vellus hair density measurement. In some embodiments, the baseline hair density measurement is assessed using photography. In some embodiments, the baseline hair density measurement is assessed by phototrichogram. In some embodiments, the baseline hair density measurement is assessed by a macrophotography analysis.

In some embodiments, the baseline measurement is a baseline SOS2 protein measurement. In some embodiments, the baseline SOS2 protein measurement comprises a baseline SOS2 protein level. In some embodiments, the baseline SOS2 protein level is indicated as a mass or percentage of SOS2 protein per sample weight. In some embodiments, the baseline SOS2 protein level is indicated as a mass or percentage of SOS2 protein per sample volume. In some embodiments, the baseline SOS2 protein level is indicated as a mass or percentage of SOS2 protein per total protein within the sample. In some embodiments, the baseline SOS2 protein measurement is a baseline tissue SOS2 protein measurement. Examples of baseline tissue SOS2 protein measurements include a baseline liver SOS2 protein measurement, a baseline kidney SOS2 protein measurement, a baseline eye SOS2 protein measurement, or a baseline adipose tissue SOS2 protein measurement. In some embodiments, the baseline SOS2 protein measurement is a baseline circulating SOS2 protein measurement. In some embodiments, the baseline SOS2 protein measurement is obtained by an assay such as an immunoassay, a colorimetric assay, or a fluorescence assay.

In some embodiments, the baseline measurement is a baseline SOS2 mRNA measurement. In some embodiments, the baseline SOS2 mRNA measurement comprises a baseline SOS2 mRNA level. In some embodiments, the baseline SOS2 mRNA level is indicated as a mass or percentage of SOS2 mRNA per sample weight. In some embodiments, the baseline SOS2 mRNA level is indicated as a mass or percentage of SOS2 mRNA per sample volume. In some embodiments, the baseline SOS2 mRNA level is indicated as a mass or percentage of SOS2 mRNA per total mRNA within the sample. In some embodiments, the baseline SOS2 mRNA level is indicated as a mass or percentage of SOS2 mRNA per total nucleic acids within the sample. In some embodiments, the baseline SOS2 mRNA level is indicated relative to another mRNA level, such as an mRNA level of a housekeeping gene, within the sample. In some embodiments, the baseline SOS2 mRNA measurement is a baseline tissue SOS2 mRNA measurement. Examples of baseline tissue SOS2 mRNA measurements include a baseline liver SOS2 mRNA measurement, a baseline kidney SOS2 mRNA measurement, a baseline eye SOS2 mRNA measurement, or a baseline adipose tissue SOS2 mRNA measurement. In some embodiments, the baseline SOS2 mRNA measurement is a baseline circulating SOS2 mRNA measurement. In some embodiments, the baseline SOS2 mRNA measurement is obtained by an assay such as a polymerase chain reaction (PCR) assay. In some embodiments, the PCR comprises quantitative PCR (qPCR). In some embodiments, the PCR comprises reverse transcription of the SOS2 mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. In some embodiments, the sample comprises serum. In some embodiments, the sample is a serum sample.

In some embodiments, the sample comprises a tissue. In some embodiments, the sample is a tissue sample. In some embodiments, the sample comprises liver tissue. In some embodiments, the sample is a liver sample. In some embodiments, the sample comprises adipose tissue. In some embodiments, the sample is an adipose sample. In some embodiments, the tissue sample comprises brown adipose tissue or white adipose tissue. In some embodiments, the sample comprises kidney tissue. In some embodiments, the sample is a kidney sample. In some embodiments, the sample comprises eye tissue. In some embodiments, the sample is an eye sample. In some embodiments, the sample comprises an eye fluid. In some embodiments, the sample comprises a hair or scalp sample. In some examples, the baseline SOS2 mRNA measurement, or the baseline SOS2 protein measurement, may be obtained in a liver, adipose, eye, or kidney sample from the patient. In some embodiments, the sample comprises cardiac tissue such as ventricular or atrial tissue. In some embodiments, the sample comprises a cerebral tissue or fluid. In some embodiments, the sample comprises a neural tissue or neural fluid. In some embodiments, the sample comprises a muscle tissue or fluid. The sample may comprise or consist of hepatocytes. The sample may comprise or consist of podocytes.

### D. Effects

In some embodiments, the composition or administration of the composition affects a measurement such as a glomerular filtration rate (GFR) or estimated glomerular filtration rate (eGFR) measurement, a creatinine measurement, a blood urea nitrogen (BUN) measurement, a proteinuria measurement, a microalbuminuria measurement, a blood urate measurement, a systolic blood pressure (SBP) measurement, a diastolic blood pressure (DBP) measurement, a intraocular pressure (IOP) measurement, a hemoglobin A1C measurement, a alanine aminotransferase (ALT) measurement, a aspartate aminotransferase (AST) measurement, a liver fat percentage (LFP) measurement, a liver fibrosis measurement, a hair count measurement, a hair thickness measurement, a hair density measurement, a SOS2 protein measurement, or a SOS2 mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, or a PCR assay. In some embodiments, the measurement is obtained by an assay such as an immunoassay, a colorimetric assay, or a fluorescence assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. In some embodiments, the reduction is measured in a second tissue sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 95% relative to the baseline measurement. In some embodiments, the measurement is decreased by 2.5%, 5%, 7.5%, 10%, 20%, 30%, 40%, 50%, 60%, 70%, 80%, 90%, or 95%, or by a range defined by any of the two aforementioned percentages.

In some embodiments, the composition increases the measurement relative to the baseline measurement. In some embodiments, the increase is measured in a second tissue sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a GFR or eGFR measurement. In some embodiments, the measurement is a GFR measurement. In some embodiments, the measurement is a eGFR measurement. The GFR or eGFR measurement may be indicated in units of volume per time (e.g. mL/min). The GFR measurement may be obtained using a clearance measurement such as a creatinine clearance measurement. The GFR may also be determined by injecting insulin, sinistrin, a radioactive tracer, or cystatin C, and determining a clearance rate. The eGFR measurement may be also be obtained using a clearance estimate such as an estimation of serum creatinine clearance. The GFR or eGFR may be 100-130 mL/min/1.73 m², 90-100 mL/min/1.73 m². The GFR or eGFR may be below 90 or 100 mL/min/1.73 m². The GFR or eGFR may be indicative of normal kidney function, CKD1, CKD2, CKD3, CKD4, or CKD5, as indicated by a kidney function index.

In some embodiments, the composition increases the GFR measurement relative to the baseline GFR or eGFR measurement. In some embodiments, the composition increases the eGFR measurement relative to the baseline GFR or eGFR measurement. In some embodiments, the increase is measured directly in the subject after administering the composition to the subject. In some embodiments, the GFR or eGFR measurement is increased by about 2.5% or more, about 5% or more, or about 7.5% or more, relative to the baseline GFR or eGFR measurement. In some embodiments, the GFR or eGFR measurement is increased by about 10% or more, relative to the baseline GFR or eGFR measurement. In some embodiments, the GFR or eGFR measurement is increased by about 20% or more, about 30% or more, about 40% or more, about 50% or more, about 60% or more, about 70% or more, about 80% or more, about 90% or more, relative to the baseline GFR or eGFR measurement. In some embodiments, the GFR or eGFR measurement is increased by about 100% or more, increased by about 250% or more, increased by about 500% or more, increased by about 750% or more, or increased by about 1000% or more, relative to the baseline GFR or eGFR measurement. In some embodiments, the GFR or eGFR measurement is increased by no more than about 2.5%, no more than about 5%, or no more than about 7.5%, relative to the baseline GFR or eGFR measurement. In some embodiments, the GFR or eGFR measurement is increased by no more than about 10%, relative to the baseline GFR or eGFR measurement. In some embodiments, the GFR or eGFR measurement is increased by no more than about 20%, no more than about 30%, no more than about 40%, no more than about 50%, no more than about 60%, no more than about 70%, no more than about 80%, no more than about 90%, or no more than about 100% relative to the baseline GFR or eGFR measurement. In some embodiments, the GFR or eGFR measurement is increased by no more than about 100%, increased by no more than about 250%, increased by no more than about 500%, increased by no more than about 750%, or increased by no more than about 1000%, relative to the baseline GFR or eGFR measurement. In some embodiments, the GFR or eGFR measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a creatinine measurement. In some embodiments, the creatinine measurement is a creatinine concentration. In some embodiments, the creatinine measurement is a circulating (e.g. serum or plasma) creatinine measurement. In some embodiments, the creatinine measurement is a urine creatinine measurement. In some embodiments, the creatinine measurement is obtained by an assay such as an immunoassay, a colorimetric assay, or a fluorescence assay. The circulating creatinine measurement may be about 0.5-1.3 mg/dL. The circulating creatinine measurement may be above 1.3 mg/dL. The circulating creatinine measurement may be within, above, or below a reference range. The urine creatinine measurement may be within, above, or below a reference range.

In some embodiments, the composition reduces the creatinine measurement relative to the baseline creatinine measurement. In some embodiments, the reduction is measured in a second fluid sample obtained from the subject after administering the composition to the subject. In some embodiments, the reduction is measured directly in the subject after administering the composition to the subject. In some embodiments, the creatinine measurement is decreased by about 2.5% or more, about 5% or more, or about 7.5% or more, relative to the baseline creatinine measurement. In some embodiments, the creatinine measurement is decreased by about 10% or more, relative to the baseline creatinine measurement. In some embodiments, the creatinine measurement is decreased by about 20% or more, about 30% or more, about 40% or more, about 50% or more, about 60% or more, about 70% or more, about 80% or more, about 90% or more, relative to the baseline creatinine measurement. In some embodiments, the creatinine measurement is decreased by no more than about 2.5%, no more than about 5%, or no more than about 7.5%, relative to the baseline creatinine measurement. In some embodiments, the creatinine measurement is decreased by no more than about 10%, relative to the baseline creatinine measurement. In some embodiments, the creatinine measurement is decreased by no more than about 20%, no more than about 30%, no more than about 40%, no more than about 50%, no more than about 60%, no more than about 70%, no more than about 80%, or no more than about 90% relative to the baseline creatinine measurement. In some embodiments, the creatinine measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a blood urea nitrogen (BUN) measurement. In some embodiments, the BUN measurement is a BUN concentration. In some embodiments, the BUN measurement is a circulating BUN measurement. In some embodiments, the BUN measurement is obtained by an assay such as an immunoassay, a colorimetric assay, or a fluorescence assay. In some embodiments, the BUN is 6-20 mg/dL. In some embodiments, the BUN is over 20 mg/dL. A normal BUN range is 6-20 mg/dL. In some embodiments, the measurement is a BUN/creatinine ratio.

In some embodiments, the composition reduces the BUN measurement relative to the baseline BUN measurement. In some embodiments, the reduction is measured in a second blood sample obtained from the subject after administering the composition to the subject. In some embodiments, the reduction is measured directly in the subject after administering the composition to the subject. In some embodiments, the BUN measurement is decreased by about 2.5% or more, about 5% or more, or about 7.5% or more, relative to the baseline BUN measurement. In some embodiments, the BUN measurement is decreased by about 10% or more, relative to the baseline BUN measurement. In some embodiments, the BUN measurement is decreased by about 20% or more, about 30% or more, about 40% or more, about 50% or more, about 60% or more, about 70% or more, about 80% or more, about 90% or more, relative to the baseline BUN measurement. In some embodiments, the BUN measurement is decreased by no more than about 2.5%, no more than about 5%, or no more than about 7.5%, relative to the baseline BUN measurement. In some embodiments, the BUN measurement is decreased by no more than about 10%, relative to the baseline BUN measurement. In some embodiments, the BUN measurement is decreased by no more than about 20%, no more than about 30%, no more than about 40%, no more than about 50%, no more than about 60%, no more than about 70%, no more than about 80%, or no more than about 90% relative to the baseline BUN measurement. In some embodiments, the BUN measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a proteinuria measurement. The proteinuria measurement may be indicated as a concentration, a ratio, or a mass/unit time (e.g. mg/mmol urine, protein/creatinine, or mg protein/hr). In some embodiments, the proteinuria measurement includes a proteinuria concentration. In some embodiments, the proteinuria measurement is a urine proteinuria measurement. In some embodiments, the proteinuria measurement is obtained by an assay such as an immunoassay, a colorimetric assay, or a fluorescence assay. In some embodiments, the proteinuria measurement is indicative of proteinuria in the subject. In some embodiments, the proteinuria measurement is indicative of a lack of proteinuria in the subject. In some embodiments, the measurement is a urine protein/creatinine ratio.

In some embodiments, the composition reduces the proteinuria measurement relative to the baseline proteinuria measurement. In some embodiments, the reduction is measured in a second fluid sample obtained from the subject after administering the composition to the subject. In some embodiments, the reduction is measured directly in the subject after administering the composition to the subject. In some embodiments, the proteinuria measurement is decreased by about 2.5% or more, about 5% or more, or about 7.5% or more, relative to the baseline proteinuria measurement. In some embodiments, the proteinuria measurement is decreased by about 10% or more, relative to the baseline proteinuria measurement. In some embodiments, the proteinuria measurement is decreased by about 20% or more, about 30% or more, about 40% or more, about 50% or more, about 60% or more, about 70% or more, about 80% or more, about 90% or more, relative to the baseline proteinuria measurement. In some embodiments, the proteinuria measurement is decreased by no more than about 2.5%, no more than about 5%, or no more than about 7.5%, relative to the baseline proteinuria measurement. In some embodiments, the proteinuria measurement is decreased by no more than about 10%, relative to the baseline proteinuria measurement. In some embodiments, the proteinuria measurement is decreased by no more than about 20%, no more than about 30%, no more than about 40%, no more than about 50%, no more than about 60%, no more than about 70%, no more than about 80%, or no more than about 90% relative to the baseline proteinuria measurement. In some embodiments, the proteinuria measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a microalbuminuria measurement. The microalbuminuria measurement may be indicated as a concentration, a ratio, or a mass/unit time (e.g. mg/mmol urine, albumin/creatinine, or mg albumin/hr). In some embodiments, the microalbuminuria measurement includes a microalbuminuria concentration. In some embodiments, the microalbuminuria measurement is a urine microalbuminuria measurement. In some embodiments, the microalbuminuria measurement is obtained by an assay such as an immunoassay, a colorimetric assay, or a fluorescence assay. In some embodiments, the microalbuminuria measurement is indicative of microalbuminuria in the subject. In some embodiments, the microalbuminuria measurement is indicative of a lack of microalbuminuria in the subject. In some embodiments, the measurement is a urine albumin/creatinine ratio. The microalbuminuria measurement may include a microalbuminuria measurement within a range or amount defined in Table 1.

In some embodiments, the composition reduces the microalbuminuria measurement relative to the baseline microalbuminuria measurement. In some embodiments, the reduction is measured in a second fluid sample obtained from the subject after administering the composition to the subject. In some embodiments, the reduction is measured directly in the subject after administering the composition to the subject. In some embodiments, the microalbuminuria measurement is decreased by about 2.5% or more, about 5% or more, or about 7.5% or more, relative to the baseline microalbuminuria measurement. In some embodiments, the microalbuminuria measurement is decreased by about 10% or more, relative to the baseline microalbuminuria measurement. In some embodiments, the microalbuminuria measurement is decreased by about 20% or more, about 30% or more, about 40% or more, about 50% or more, about 60% or more, about 70% or more, about 80% or more, about 90% or more, relative to the baseline microalbuminuria measurement. In some embodiments, the microalbuminuria measurement is decreased by no more than about 2.5%, no more than about 5%, or no more than about 7.5%, relative to the baseline microalbuminuria measurement. In some embodiments, the microalbuminuria measurement is decreased by no more than about 10%, relative to the baseline microalbuminuria measurement. In some embodiments, the microalbuminuria measurement is decreased by no more than about 20%, no more than about 30%, no more than about 40%, no more than about 50%, no more than about 60%, no more than about 70%, no more than about 80%, or no more than about 90% relative to the baseline microalbuminuria measurement. In some embodiments, the microalbuminuria measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a blood urate measurement. In some embodiments, the blood urate measurement is a blood urate concentration. In some embodiments, the blood urate measurement is obtained by an assay such as an immunoassay, a colorimetric assay, or a fluorescence assay. In some embodiments, the blood urate measurement is indicative of hyperuricemia. In some embodiments, the blood urate measurement is indicative of a lack of hyperuricemia. For example, the serum uric acid measurement may be 6 mg/dL or less, 7 mg/dL or less, or 5.5 mg/dL or less.

In some embodiments, the composition reduces the blood urate measurement relative to the baseline blood urate measurement. In some embodiments, the reduction is measured in a second blood sample obtained from the subject after administering the composition to the subject. In some embodiments, the reduction is measured directly in the subject after administering the composition to the subject. In some embodiments, the blood urate measurement is decreased by about 2.5% or more, about 5% or more, or about 7.5% or more, relative to the baseline blood urate measurement. In some embodiments, the blood urate measurement is decreased by about 10% or more, relative to the baseline blood urate measurement. In some embodiments, the blood urate measurement is decreased by about 20% or more, about 30% or more, about 40% or more, about 50% or more, about 60% or more, about 70% or more, about 80% or more, about 90% or more, relative to the baseline blood urate measurement. In some embodiments, the blood urate measurement is decreased by no more than about 2.5%, no more than about 5%, or no more than about 7.5%, relative to the baseline blood urate measurement. In some embodiments, the blood urate measurement is decreased by no more than about 10%, relative to the baseline blood urate measurement. In some embodiments, the blood urate measurement is decreased by no more than about 20%, no more than about 30%, no more than about 40%, no more than about 50%, no more than about 60%, no more than about 70%, no more than about 80%, or no more than about 90% relative to the baseline blood urate measurement. In some embodiments, the blood urate measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a systolic blood (SBP) pressure measurement. In some embodiments, the SBP measurement is measured in mm of mercury (mm Hg). In some embodiments, the SBP measurement is obtained with a sphygmomanometer. The SBP measurement may be indicative of hypertension. The SBP measurement may be indicative of normal blood pressure. The SBP measurement may include a cerebral SBP measurement.

In some embodiments, the composition reduces the SBP measurement relative to the baseline SBP measurement. In some embodiments, the reduction is measured directly in the subject after administering the composition to the subject. In some embodiments, the SBP measurement is decreased by about 2.5% or more, about 5% or more, or about 7.5% or more, relative to the baseline SBP measurement. In some embodiments, the SBP measurement is decreased by about 10% or more, relative to the baseline SBP measurement. In some embodiments, the SBP measurement is decreased by about 20% or more, about 30% or more, about 40% or more, about 50% or more, about 60% or more, about 70% or more, about 80% or more, about 90% or more, relative to the baseline SBP measurement. In some embodiments, the SBP measurement is decreased by no more than about 2.5%, no more than about 5%, or no more than about 7.5%, relative to the baseline SBP measurement. In some embodiments, the SBP measurement is decreased by no more than about 10%, relative to the baseline SBP measurement. In some embodiments, the SBP measurement is decreased by no more than about 20%, no more than about 30%, no more than about 40%, no more than about 50%, no more than about 60%, no more than about 70%, no more than about 80%, or no more than about 90% relative to the baseline SBP measurement. In some embodiments, the SBP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a diastolic blood (DBP) pressure measurement. In some embodiments, the DBP measurement is measured in mm of mercury (mm Hg). In some embodiments, the DBP measurement is obtained with a sphygmomanometer. The DBP measurement may be indicative of hypertension. The DBP measurement may be indicative of normal blood pressure. The DBP measurement may include a cerebral DBP measurement.

In some embodiments, the composition reduces the DBP measurement relative to the baseline DBP measurement. In some embodiments, the reduction is measured directly in the subject after administering the composition to the subject. In some embodiments, the DBP measurement is decreased by about 2.5% or more, about 5% or more, or about 7.5% or more, relative to the baseline DBP measurement. In some embodiments, the DBP measurement is decreased by about 10% or more, relative to the baseline DBP measurement. In some embodiments, the DBP measurement is decreased by about 20% or more, about 30% or more, about 40% or more, about 50% or more, about 60% or more, about 70% or more, about 80% or more, about 90% or more, relative to the baseline DBP measurement. In some embodiments, the DBP measurement is decreased by no more than about 2.5%, no more than about 5%, or no more than about 7.5%, relative to the baseline DBP measurement. In some embodiments, the DBP measurement is decreased by no more than about 10%, relative to the baseline DBP measurement. In some embodiments, the DBP measurement is decreased by no more than about 20%, no more than about 30%, no more than about 40%, no more than about 50%, no more than about 60%, no more than about 70%, no more than about 80%, or no more than about 90% relative to the baseline DBP measurement. In some embodiments, the DBP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a intraocular pressure (IOP) measurement. The IOP may be measured using a tonometer. The IOP measurement may be in millimeters of mercury (mmHg). The IOP measurement may be indicative of a normal IOP. The IOP measurement may be indicative of abnormal or high IOP.

In some embodiments, the composition reduces the IOP measurement relative to the baseline IOP measurement. In some embodiments, the reduction is measured directly in the subject after administering the composition to the subject. In some embodiments, the IOP measurement is decreased by about 2.5% or more, about 5% or more, or about 7.5% or more, relative to the baseline IOP measurement. In some embodiments, the IOP measurement is decreased by about 10% or more, relative to the baseline IOP measurement. In some embodiments, the IOP measurement is decreased by about 20% or more, about 30% or more, about 40% or more, about 50% or more, about 60% or more, about 70% or more, about 80% or more, about 90% or more, relative to the baseline IOP measurement. In some embodiments, the IOP measurement is decreased by no more than about 2.5%, no more than about 5%, or no more than about 7.5%, relative to the baseline IOP measurement. In some embodiments, the IOP measurement is decreased by no more than about 10%, relative to the baseline IOP measurement. In some embodiments, the IOP measurement is decreased by no more than about 20%, no more than about 30%, no more than about 40%, no more than about 50%, no more than about 60%, no more than about 70%, no more than about 80%, or no more than about 90% relative to the baseline IOP measurement. In some embodiments, the IOP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a hemoglobin A1C measurement. In some embodiments, the hemoglobin A1C measurement is a hemoglobin A1C concentration. In some embodiments, the hemoglobin A1C measurement is a circulating hemoglobin A1C measurement. In some embodiments, the hemoglobin A1C measurement is obtained by an assay such as an immunoassay, a colorimetric assay, a fluorescence assay, or HPLC. The hemoglobin A1C measurement may be indicative of a healthy normal A1C measurement. The hemoglobin A1C measurement may be indicative of diabetes. The hemoglobin A1C measurement may be indicative of pre-diabetes.

In some embodiments, the composition reduces the hemoglobin A1C measurement relative to the baseline hemoglobin A1C measurement. In some embodiments, the reduction is measured in a second fluid sample obtained from the subject after administering the composition to the subject. In some embodiments, the reduction is measured directly in the subject after administering the composition to the subject. In some embodiments, the hemoglobin A1C measurement is decreased by about 2.5% or more, about 5% or more, or about 7.5% or more, relative to the baseline hemoglobin A1C measurement. In some embodiments, the hemoglobin A1C measurement is decreased by about 10% or more, relative to the baseline hemoglobin A1C measurement. In some embodiments, the hemoglobin A1C measurement is decreased by about 20% or more, about 30% or more, about 40% or more, about 50% or more, about 60% or more, about 70% or more, about 80% or more, about 90% or more, relative to the baseline hemoglobin A1C measurement. In some embodiments, the hemoglobin A1C measurement is decreased by no more than about 2.5%, no more than about 5%, or no more than about 7.5%, relative to the baseline hemoglobin A1C measurement. In some embodiments, the hemoglobin A1C measurement is decreased by no more than about 10%, relative to the baseline hemoglobin A1C measurement. In some embodiments, the hemoglobin A1C measurement is decreased by no more than about 20%, no more than about 30%, no more than about 40%, no more than about 50%, no more than about 60%, no more than about 70%, no more than about 80%, or no more than about 90% relative to the baseline hemoglobin A1C measurement. In some embodiments, the hemoglobin A1C measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a liver enzyme measurement. In some embodiments, the liver enzyme measurement is an alanine aminotransferase (ALT) measurement. In some embodiments, the liver enzyme measurement is an aspartate aminotransferase (AST) measurement. In some embodiments, the liver enzyme measurement comprises an ALT/AST ratio, or comprises an AST/ALT ratio.

In some embodiments, the measurement is a alanine aminotransferase (ALT) measurement. In some embodiments, the ALT measurement is a ALT concentration (for example, Units/dL). In some embodiments, the ALT measurement is a circulating ALT measurement, for example, a blood, serum, or plasma ALT measurement. In some embodiments, the ALT measurement is obtained by an assay such as an immunoassay, a colorimetric assay, or a fluorescence assay. In some embodiments, the ALT measurement is within a reference range. In some embodiments, the ALT measurement is above the reference range.

In some embodiments, the composition reduces the ALT measurement relative to the baseline ALT measurement. In some embodiments, the reduced ALT is measured in a second blood sample, plasma sample, or serum sample obtained from the subject after administering the composition to the subject. In some embodiments, the ALT measurement is decreased by about 2.5% or more, about 5% or more, or about 7.5% or more, relative to the baseline ALT measurement. In some embodiments, the ALT measurement is decreased by about 10% or more, relative to the baseline ALT measurement. In some embodiments, the ALT measurement is decreased by about 20% or more, about 30% or more, about 40% or more, about 50% or more, about 60% or more, about 70% or more, about 80% or more, about 90% or more, relative to the baseline ALT measurement. In some embodiments, the ALT measurement is decreased by no more than about 2.5%, no more than about 5%, or no more than about 7.5%, relative to the baseline ALT measurement. In some embodiments, the ALT measurement is decreased by no more than about 10%, relative to the baseline ALT measurement. In some embodiments, the ALT measurement is decreased by no more than about 20%, no more than about 30%, no more than about 40%, no more than about 50%, no more than about 60%, no more than about 70%, no more than about 80%, or no more than about 90% relative to the baseline ALT measurement. In some embodiments, the ALT measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a aspartate aminotransferase (AST) measurement. In some embodiments, the AST measurement is a AST concentration (for example, Units/L). In some embodiments, the AST measurement is a circulating AST measurement, for example, a blood, serum, or plasma AST measurement. In some embodiments, the AST measurement is obtained by an assay such as an immunoassay, a colorimetric assay, or a fluorescence assay. In some embodiments, the AST measurement is within a reference range. In some embodiments, the AST measurement is above the reference range. In some embodiments, the AST measurement is below the reference range.

In some embodiments, the composition reduces the AST measurement relative to the baseline AST measurement. In some embodiments, the reduced AST is measured in a second blood sample, plasma sample, or serum sample obtained from the subject after administering the composition to the subject. In some embodiments, the AST measurement is decreased by about 2.5% or more, about 5% or more, or about 7.5% or more, relative to the baseline AST measurement. In some embodiments, the AST measurement is decreased by about 10% or more, relative to the baseline AST measurement. In some embodiments, the AST measurement is decreased by about 20% or more, about 30% or more, about 40% or more, about 50% or more, about 60% or more, about 70% or more, about 80% or more, about 90% or more, relative to the baseline AST measurement. In some embodiments, the AST measurement is decreased by no more than about 2.5%, no more than about 5%, or no more than about 7.5%, relative to the baseline AST measurement. In some embodiments, the AST measurement is decreased by no more than about 10%, relative to the baseline AST measurement. In some embodiments, the AST measurement is decreased by no more than about 20%, no more than about 30%, no more than about 40%, no more than about 50%, no more than about 60%, no more than about 70%, no more than about 80%, no more than about 90% relative to the baseline AST measurement. In some embodiments, the AST measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a liver steatosis measurement. In some embodiments, the liver steatosis measurement is a liver fat percentage (LFP) measurement. In some embodiments, the measurement is a LFP measurement. In some embodiments, the LFP measurement is indicated as a mass/mass percentage of fat/total tissue. In some embodiments, the LFP measurement is indicated as a mass/volume percentage of fat/total tissue. In some embodiments, the LFP measurement is indicated as a volume/mass percentage of fat/total tissue. In some embodiments, the LFP measurement is indicated as a volume/volume percentage of fat/total tissue. In some embodiments, the LFP measurement is indicated as a score. In some embodiments, the LFP measurement is obtained noninvasively. In some embodiments, the LFP measurement is obtained by a medical imaging device. In some embodiments, the LFP measurement is obtained by a device such as a medical resonance imaging (MRI) device, a magnetic resonance spectroscopy device, a computed tomography device, a controlled attenuation parameter (CAP), a transient elastography device, or an ultrasound device. In some embodiments, the LFP measurement is obtained in a second liver sample. In some embodiments, the LFP measurement comprises a liver triglyceride measurement. In some embodiments, the LFP measurement is obtained by an assay such as an immunoassay, a colorimetric assay, or a fluorescence assay. In some embodiments, the LFP measurement or the LFP measurement is obtained using a scoring system upon a visual inspection of a sample such as a histological sample. In some embodiments, the LFP measurement or the LFP measurement is obtained using a stain with an affinity to fats, such as a lysochrome diazo dye.

In some embodiments, the composition reduces the LFP measurement relative to the baseline LFP measurement. In some embodiments, the reduced LFP is measured in a second liver sample obtained from the subject after administering the composition to the subject. In some embodiments, the reduced LFP is measured directly in the subject after administering the composition to the subject. In some embodiments, the LFP measurement is decreased by about 2.5% or more, about 5% or more, or about 7.5% or more, relative to the baseline LFP measurement. In some embodiments, the LFP measurement is decreased by about 10% or more, relative to the baseline LFP measurement. In some embodiments, the LFP measurement is decreased by about 20% or more, about 30% or more, about 40% or more, about 50% or more, about 60% or more, about 70% or more, about 80% or more, about 90% or more, relative to the baseline LFP measurement. In some embodiments, the LFP measurement is decreased by no more than about 2.5%, no more than about 5%, or no more than about 7.5%, relative to the baseline LFP measurement. In some embodiments, the LFP measurement is decreased by no more than about 10%, relative to the baseline LFP measurement. In some embodiments, the LFP measurement is decreased by no more than about 20%, no more than about 30%, no more than about 40%, no more than about 50%, no more than about 60%, no more than about 70%, no more than about 80%, or no more than about 90% relative to the baseline LFP measurement. In some embodiments, the LFP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a liver fibrosis measurement. In some embodiments, the liver fibrosis measurement is a liver fibrosis score (LFS). In some embodiments, the LFS comprises a score of 0, 1, 2, 3, or 4, or a range of scores defined by any two of the aforementioned numbers. In some embodiments, the LFS comprises a score of 0-4. In some embodiments, the LFS is obtained using a scoring system exemplified in Table 1. In some embodiments, the LFS measurement is obtained noninvasively. In some embodiments, the LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the LFS measurement is obtained in a second liver sample. In some embodiments, the LFS is obtained by an assay such as an immunoassay, a colorimetric assay, or a fluorescence assay. In some embodiments, the LFS is obtained using one or more indirect markers or measures of liver fibrosis such as an aspartate aminotransferase-to-platelet ratio index (APRI), a Fibrosis-4 (FIB-4) index, a FibroIndex, a Forns Index, a Hepascore, or a FibroTest. In some embodiments, the LFS is obtained using one or more indirect markers or measures of liver fibrosis such as a FIBROSpect test or a FIBROSpect II test. In some embodiments, the LFS is obtained by RT-qPCR or RNA sequencing of one or more fibrosis-related genes such as a collagen gene. In some embodiments, the LFS or the LFS is obtained using a scoring system upon a visual inspection of a sample such as a histological sample. In some embodiments, the LFS or the LFS is obtained using a stain with an affinity to collagen.

In some embodiments, the composition reduces the LFS relative to the baseline LFS. In some embodiments, the reduced LFS is measured in a second liver sample obtained from the subject after administering the composition to the subject. In some embodiments, the reduced LFS is measured directly in the subject after administering the composition to the subject. In some embodiments, the LFS is decreased by 1 relative to the baseline LFS. In some embodiments, the LFS is decreased by 2 relative to the baseline LFS. In some embodiments, the LFS is decreased by 3 relative to the baseline LFS. In some embodiments, the LFS is decreased by 4 relative to the baseline LFS. In some embodiments, the LFS is decreased by 1 or more, relative to the baseline LFS. In some embodiments, the LFS is decreased by 2 or more, relative to the baseline LFS. In some embodiments, the LFS is decreased by 3 more, relative to the baseline LFS. In some embodiments, the LFS is decreased by no more than 1, relative to the baseline LFS. In some embodiments, the LFS is decreased by no more than 2, relative to the baseline LFS. In some embodiments, the LFS is decreased by no more than 3, relative to the baseline LFS. In some embodiments, the LFS is decreased by no more than 4, relative to the baseline LFS. In some embodiments, the LFS is decreased by 1, 2, 3, or 4, or by a range defined by any of the two aforementioned numbers.

In some embodiments, the measurement is a hair count. In some embodiments, the hair count is a total hair count. The total hair count may include a vellus hair count and a non-vellus hair count. In some embodiments, the hair count is a vellus hair count. In some embodiments, the hair count is a non-vellus hair count. In some embodiments, the hair count is determined in an area of skin. In some embodiments, the hair count is normalized based on the area of skin. In some embodiments, the hair count is assessed using photography. In some embodiments, the hair count is assessed by phototrichogram. In some embodiments, the hair count is assessed by a macrophotography analysis.

In some embodiments, the composition increases the hair count relative to the baseline hair count. In some embodiments, the increase is measured in the subject after administering the composition to the subject. In some embodiments, the increase is measured directly on the subject after administering the composition to the subject. In some embodiments, the hair count is increased by about 2.5% or more, about 5% or more, or about 7.5% or more, relative to the baseline hair count. In some embodiments, the hair count is increased by about 10% or more, relative to the baseline hair count. In some embodiments, the hair count is increased by about 20% or more, about 30% or more, about 40% or more, about 50% or more, about 60% or more, about 70% or more, about 80% or more, about 90% or more, relative to the baseline hair count. In some embodiments, the hair count is increased by about 100% or more, increased by about 250% or more, increased by about 500% or more, increased by about 750% or more, or increased by about 1000% or more, relative to the baseline hair count. In some embodiments, the hair count is increased by no more than about 2.5%, no more than about 5%, or no more than about 7.5%, relative to the baseline hair count. In some embodiments, the hair count is increased by no more than about 10%, relative to the baseline hair count. In some embodiments, the hair count is increased by no more than about 20%, no more than about 30%, no more than about 40%, no more than about 50%, no more than about 60%, no more than about 70%, no more than about 80%, no more than about 90%, or no more than about 100% relative to the baseline hair count. In some embodiments, the hair count is increased by no more than about 100%, increased by no more than about 250%, increased by no more than about 500%, increased by no more than about 750%, or increased by no more than about 1000%, relative to the baseline hair count. In some embodiments, the hair count is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a hair thickness measurement. In some embodiments, the hair thickness measurement is determined in an area of skin. In some embodiments, the hair thickness measurement comprises a width of an individual hair. In some embodiments, the hair thickness measurement comprises widths of multiple individual hairs. In some embodiments, the hair thickness measurement comprises an average of the widths of the multiple individual hairs. In some embodiments, the hair thickness measurement comprises a median of the widths of the multiple individual hairs. The hair thickness measurement may include a vellus hair thickness measurement. The hair thickness measurement may include a non-vellus hair thickness measurement. In some embodiments, the hair thickness measurement is assessed using photography. In some embodiments, the hair thickness measurement is assessed by phototrichogram. In some embodiments, the hair thickness measurement is assessed by a macrophotography analysis.

In some embodiments, the composition increases the hair thickness measurement relative to the baseline hair thickness measurement. In some embodiments, the increase is measured in a second tissue sample (e.g. a skin sample as described herein) obtained from the subject after administering the composition to the subject. In some embodiments, the increase is measured directly on the subject after administering the composition to the subject. In some embodiments, the hair thickness measurement is increased by about 2.5% or more, about 5% or more, or about 7.5% or more, relative to the baseline hair thickness measurement. In some embodiments, the hair thickness measurement is increased by about 10% or more, relative to the baseline hair thickness measurement. In some embodiments, the hair thickness measurement is increased by about 20% or more, about 30% or more, about 40% or more, about 50% or more, about 60% or more, about 70% or more, about 80% or more, about 90% or more, relative to the baseline hair thickness measurement. In some embodiments, the hair thickness measurement is increased by about 100% or more, increased by about 250% or more, increased by about 500% or more, increased by about 750% or more, or increased by about 1000% or more, relative to the baseline hair thickness measurement. In some embodiments, the hair thickness measurement is increased by no more than about 2.5%, no more than about 5%, or no more than about 7.5%, relative to the baseline hair thickness measurement. In some embodiments, the hair thickness measurement is increased by no more than about 10%, relative to the baseline hair thickness measurement. In some embodiments, the hair thickness measurement is increased by no more than about 20%, no more than about 30%, no more than about 40%, no more than about 50%, no more than about 60%, no more than about 70%, no more than about 80%, no more than about 90%, or no more than about 100% relative to the baseline hair thickness measurement. In some embodiments, the hair thickness measurement is increased by no more than about 100%, increased by no more than about 250%, increased by no more than about 500%, increased by no more than about 750%, or increased by no more than about 1000%, relative to the baseline hair thickness measurement. In some embodiments, the hair thickness measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a hair density measurement. In some embodiments, the hair density measurement is determined in an area of skin. In some embodiments, the hair density measurement comprises a number of hair in the area of skin. In some embodiments, the hair density measurement comprises the number of hair in the area of skin divided by the area of skin. The hair density measurement may include a vellus hair density measurement. The hair density measurement may include a non-vellus hair density measurement. In some embodiments, the hair density measurement is assessed using photography. In some embodiments, the hair density measurement is assessed by phototrichogram. In some embodiments, the hair density measurement is assessed by a macrophotography analysis.

In some embodiments, the composition increases the hair density measurement relative to the baseline hair density measurement. In some embodiments, the increase is measured in a second tissue sample (e.g. a skin sample as described herein) obtained from the subject after administering the composition to the subject. In some embodiments, the increase is measured directly on the subject after administering the composition to the subject. In some embodiments, the hair density measurement is increased by about 2.5% or more, about 5% or more, or about 7.5% or more, relative to the baseline hair density measurement. In some embodiments, the hair density measurement is increased by about 10% or more, relative to the baseline hair density measurement. In some embodiments, the hair density measurement is increased by about 20% or more, about 30% or more, about 40% or more, about 50% or more, about 60% or more, about 70% or more, about 80% or more, about 90% or more, relative to the baseline hair density measurement. In some embodiments, the hair density measurement is increased by about 100% or more, increased by about 250% or more, increased by about 500% or more, increased by about 750% or more, or increased by about 1000% or more, relative to the baseline hair density measurement. In some embodiments, the hair density measurement is increased by no more than about 2.5%, no more than about 5%, or no more than about 7.5%, relative to the baseline hair density measurement. In some embodiments, the hair density measurement is increased by no more than about 10%, relative to the baseline hair density measurement. In some embodiments, the hair density measurement is increased by no more than about 20%, no more than about 30%, no more than about 40%, no more than about 50%, no more than about 60%, no more than about 70%, no more than about 80%, no more than about 90%, or no more than about 100% relative to the baseline hair density measurement. In some embodiments, the hair density measurement is increased by no more than about 100%, increased by no more than about 250%, increased by no more than about 500%, increased by no more than about 750%, or increased by no more than about 1000%, relative to the baseline hair density measurement. In some embodiments, the hair density measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a SOS2 protein measurement. In some embodiments, the SOS2 protein measurement comprises a SOS2 protein level. In some embodiments, the SOS2 protein level is indicated as a mass or percentage of SOS2 protein per sample weight. In some embodiments, the SOS2 protein level is indicated as a mass or percentage of SOS2 protein per sample volume. In some embodiments, the SOS2 protein level is indicated as a mass or percentage of SOS2 protein per total protein within the sample. In some embodiments, the SOS2 protein measurement is a tissue SOS2 protein measurement. Examples of tissue SOS2 protein measurements include a liver SOS2 protein measurement, a kidney SOS2 protein measurement, a eye SOS2 protein measurement, or a adipose tissue SOS2 protein measurement. In some embodiments, the SOS2 protein measurement is a circulating SOS2 protein measurement. In some embodiments, the SOS2 protein measurement is obtained by an assay such as an immunoassay, a colorimetric assay, or a fluorescence assay.

In some embodiments, the composition reduces the SOS2 protein measurement relative to the baseline SOS2 protein measurement. In some embodiments, the reduction is measured in a second sample (e.g. a tissue sample such as liver, kidney, adipose, or eye tissue) obtained from the subject after administering the composition to the subject. In some embodiments, the SOS2 protein measurement is decreased by about 2.5% or more, about 5% or more, or about 7.5% or more, relative to the baseline SOS2 protein measurement. In some embodiments, the SOS2 protein measurement is decreased by about 10% or more, relative to the baseline SOS2 protein measurement. In some embodiments, the SOS2 protein measurement is decreased by about 20% or more, about 30% or more, about 40% or more, about 50% or more, about 60% or more, about 70% or more, about 80% or more, about 90% or more, relative to the baseline SOS2 protein measurement. In some embodiments, the SOS2 protein measurement is decreased by no more than about 2.5%, no more than about 5%, or no more than about 7.5%, relative to the baseline SOS2 protein measurement. In some embodiments, the SOS2 protein measurement is decreased by no more than about 10%, relative to the baseline SOS2 protein measurement. In some embodiments, the SOS2 protein measurement is decreased by no more than about 20%, no more than about 30%, no more than about 40%, no more than about 50%, no more than about 60%, no more than about 70%, no more than about 80%, or no more than about 90% relative to the baseline SOS2 protein measurement. In some embodiments, the SOS2 protein measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a SOS2 mRNA measurement. In some embodiments, the SOS2 mRNA measurement comprises a SOS2 mRNA level. In some embodiments, the SOS2 mRNA level is indicated as a mass or percentage of SOS2 mRNA per sample weight. In some embodiments, the SOS2 mRNA level is indicated as a mass or percentage of SOS2 mRNA per sample volume. In some embodiments, the SOS2 mRNA level is indicated as a mass or percentage of SOS2 mRNA per total mRNA within the sample. In some embodiments, the SOS2 mRNA level is indicated as a mass or percentage of SOS2 mRNA per total nucleic acids within the sample. In some embodiments, the SOS2 mRNA level is indicated relative to another mRNA level, such as an mRNA level of a housekeeping gene, within the sample. In some embodiments, the SOS2 mRNA measurement is a tissue SOS2 mRNA measurement. Examples of tissue SOS2 mRNA measurements include a liver SOS2 mRNA measurement, a kidney SOS2 mRNA measurement, a eye SOS2 mRNA measurement, or a adipose tissue SOS2 mRNA measurement. In some embodiments, the SOS2 mRNA measurement is a circulating SOS2 mRNA measurement. In some embodiments, the SOS2 mRNA measurement is obtained by an assay such as a polymerase chain reaction (PCR) assay. In some embodiments, the PCR comprises quantitative PCR (qPCR). In some embodiments, the PCR comprises reverse transcription of the SOS2 mRNA.

In some embodiments, the composition reduces the SOS2 mRNA measurement relative to the baseline SOS2 mRNA measurement. In some embodiments, the reduction is measured in a second sample (e.g. a tissue sample such as liver, kidney, adipose, or eye tissue) obtained from the subject after administering the composition to the subject. The second sample may comprise or consist of hepatocytes. The sample may comprise or consist of podocytes. In some embodiments, the SOS2 mRNA measurement is decreased by about 2.5% or more, about 5% or more, or about 7.5% or more, relative to the baseline SOS2 mRNA measurement. In some embodiments, the SOS2 mRNA measurement is decreased by about 10% or more, relative to the baseline SOS2 mRNA measurement. In some embodiments, the SOS2 mRNA measurement is decreased by about 20% or more, about 30% or more, about 40% or more, about 50% or more, about 60% or more, about 70% or more, about 80% or more, about 90% or more, relative to the baseline SOS2 mRNA measurement. In some embodiments, the SOS2 mRNA measurement is decreased by no more than about 2.5%, no more than about 5%, or no more than about 7.5%, relative to the baseline SOS2 mRNA measurement. In some embodiments, the SOS2 mRNA measurement is decreased by no more than about 10%, relative to the baseline SOS2 mRNA measurement. In some embodiments, the SOS2 mRNA measurement is decreased by no more than about 20%, no more than about 30%, no more than about 40%, no more than about 50%, no more than about 60%, no more than about 70%, no more than about 80%, or no more than about 90% relative to the baseline SOS2 mRNA measurement. In some embodiments, the SOS2 mRNA measurement is decreased by 2.5%, 5%, 7.5%, 10%, 20%, 30%, 40%, 50%, 60%, 70%, 80%, or 90%, or by a range defined by any of the two aforementioned percentages.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

Some embodiments refer to nucleic acid sequence information. In some embodiments, any uracil (U) may be interchanged with any thymine (T), and vice versa. For example, in an siRNA with a nucleic acid sequence comprising one or more Us, in some embodiments any of the Us may be replaced with Ts. Similarly, in an siRNA with a nucleic acid sequence comprising one or more Ts, in some embodiments any of the Ts may be replaced with Us. In some embodiments, an oligonucleotide such as an siRNA disclosed herein comprises or consists of RNA. In some embodiments, the oligonucleotide may comprise or consist of DNA.

The section headings used herein are for organizational purposes only and are not to be construed as limiting the subject matter described.

### VI. Numbered Embodiments

Some aspects include or relate to any of the following embodiments:

1. A composition comprising an oligonucleotide that targets SOS2 and when administered to a subject in an effective amount increases an estimated glomerular filtration rate, or decreases a creatinine, blood urea nitrogen, proteinuria or microalbuminuria measurement.

2. The composition of embodiment 1, wherein the estimated glomerular filtration rate is increased, or the creatinine, blood urea nitrogen, proteinuria or microalbuminuria measurement is decreased, by about 10% or more, as compared to prior to administration.

3. A composition comprising an oligonucleotide that targets SOS2 and when administered to a subject in an effective amount decreases a blood urate measurement.

4. The composition of embodiment 3, wherein the blood urate measurement is decreased by about 10% or more, as compared to prior to administration.

5. A composition comprising an oligonucleotide that targets SOS2 and when administered to a subject in an effective amount decreases a systolic or diastolic blood pressure measurement.

6. The composition of embodiment 5, wherein the systolic or diastolic blood pressure measurement is decreased by about 10% or more, as compared to prior to administration.

7. A composition comprising an oligonucleotide that targets SOS2 and when administered to a subject in an effective amount decreases an intraocular pressure measurement.

8. The composition of embodiment 7, wherein the intraocular pressure measurement is decreased by about 10% or more, as compared to prior to administration.

9. A composition comprising an oligonucleotide that targets SOS2 and when administered to a subject in an effective amount decreases a hemoglobin A1C measurement.

10. The composition of embodiment 9, wherein the hemoglobin A1C measurement is decreased by about 10% or more, as compared to prior to administration.

11. A composition comprising an oligonucleotide that targets SOS2 and when administered to a subject in an effective amount decreases an alanine aminotransferase, aspartate aminotransferase, or liver fat percentage measurement.

12. The composition of embodiment 11, wherein the alanine aminotransferase, aspartate aminotransferase, or liver fat percentage measurement is decreased by about 10% or more, as compared to prior to administration.

13. The composition of any one of embodiments 1-12, wherein the oligonucleotide comprises a small interfering RNA (siRNA) comprising a sense strand and an antisense strand.

14. The composition of embodiment 13, wherein the sense strand is 12-30 nucleosides in length.

15. The composition of embodiment 13 or 14, wherein the antisense strand is 12-30 nucleosides in length.

16. A composition comprising an oligonucleotide that inhibits the expression of SOS2 wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 1.

17. The composition of any one of embodiments 1-12, wherein the oligonucleotide comprises an antisense oligonucleotide (ASO).

18. A composition comprising an oligonucleotide that inhibits the expression of SOS2 wherein the oligonucleotide comprises an ASO that is complementary to a nucleoside sequence comprising about 12-30 contiguous nucleosides of SEQ ID NO: 1.

19. The composition of embodiment 17 or 18, wherein the ASO is 12-30 nucleosides in length.

20. The composition of any one of embodiments 1-19, wherein the oligonucleotide comprises a modified internucleoside linkage.

21. The composition of embodiment 20, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

22. The composition of embodiment 20, wherein the modified internucleoside linkage comprises one or more phosphorothioate linkages.

23. The composition of any one of embodiments 1-22, wherein the oligonucleotide comprises 2, 3, 4, 5, 6, 7, 8, 9, 10, 11, 12, 13, 14, 15, 16, 17, 18, 19, or 20 modified internucleoside linkages.

24. The composition of any one of embodiments 1-23, wherein the oligonucleotide comprises a modified nucleoside.

25. The composition of embodiment 24, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

26. The composition of embodiment 24, wherein the modified nucleoside comprises a LNA.

27. The composition of embodiment 24, wherein the modified nucleoside comprises a 2′,4′ constrained ethyl nucleic acid.

28. The composition of embodiment 24, wherein the modified nucleoside comprises a 2′-nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

29. The composition of embodiment 24, wherein the modified nucleoside comprises one or more 2′fluoro modified nucleosides.

30. The composition of embodiment 24, wherein the modified nucleoside comprises a 2′ modified nucleoside.

31. The composition of embodiment 24, wherein the oligonucleotide comprises a lipid attached at a 3′ or 5′ terminus of the oligonucleotide.

32. The composition of embodiment 31, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

33. The composition of any one of embodiments 1-32, wherein the oligonucleotide comprises 2, 3, 4, 5, 6, 7, 8, 9, 10, 11, 12, 13, 14, 15, 16, 17, 18, 19, 20, or 21 modified nucleosides.

34. The composition of any one of embodiments 1-33, wherein the oligonucleotide comprises an N-acetylgalactosamine (GalNAc) ligand, an arginine-glycine-aspartic acid (RGD) peptide, or a cholesterol ligand.

35. A method of treating chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss in a subject in need thereof comprising administering to the subject a composition according to any one of embodiments 1-34.

### VI. Examples

### Example 1: Functional Variants in SOS2 Demonstrate Protective Associations Across a Spectrum of Diseases and Traits

Two low-frequency missense variants (Table 3) were evaluated in approximately 375,000 individuals from the UK Biobank cohort for associations with a variety of traits. The variants were rs72681869, a low frequency (EAF=0.01) missense variant (Pro191Arg; P191R) located in the N-terminal histone-fold domain of SOS2, and rs137961578, a rare (EAF=0.001) missense variant (Leu183Phe; L183F) also located in the N-terminal histone-fold domain. SOS2 knockout mice may have lower blood urate and aspartate amino transferase (AST) than wildtype mice. Patients with pathogenic activating mutations in SOS2 may have Noonan syndrome. SOS2 biology may relate to sex hormones and androgen sensitivity (e.g. testosterone). Based on these prior observations and novel results presented here, it was considered that both SOS2 missense variants may be loss-of-function variants that result in a decrease in the abundance or activity of the SOS2 gene product. Stepwise conditional analyses in multiple traits, as well as direct evaluation of linkage disequilibrium, confirmed that they are independent variants. Therefore, these two variants were also combined and tested in a gene burden test to increase statistical power.

TABLE 3
SOS2 genetic variants/instruments utilized in this study

| | | | Proposed | |
|---|---|---|---|---|
| rsID | Gene | Consequence | Direction | EAF |
| rs72681869 | SOS2 | missense (Pro191Arg; P191R) | ↓ | 0.01 |
| rs137961578 | SOS2 | missense (Leu183Phe; L183F) | ↓ | 0.001 |
| Burden | SOS2 | P191R and/or L183F | ↓ | 0.01 |

The analyses presented here used a logistic or linear regression model with age, sex and the first ten principal components of genetic ancestry as covariates. These analyses resulted in identification of pleiotropic associations for the SOS2 missense variants. For example, there were protective associations with multiple kidney traits. The rs72681869 (P191R) variant was significantly associated with increased estimated glomerular filtration rate (eGFR) and decreased blood creatinine, and nominally associated with decreased blood urea nitrogen (BUN) and decreased risk of chronic kidney disease (CKD) and proteinuria (Table 4A and Table 4B). The rs72681869 (P191R) variant also demonstrate a trend towards decreased risk of hypertensive CKD (OR=0.72) and diabetic nephropathy (OR=0.58). The rs137961578 (L183F) variant was independently, nominally associated with increased eGFR and decreased blood creatinine, with similar effect sizes as compared to the rs72681869 (P191R) variant. The rs137961578 (L183F) variant demonstrated a trend towards decreased risk of overall CKD (OR=0.93) and hypertensive CKD (OR=0.24). The burden test was significantly associated with increased eGFR and decreased blood creatinine, suggestively associated with decreased BUN and nominally associated with decreased risk of overall CKD (OR=0.86), hypertensive CKD (OR=0.66) and proteinuria (OR=0.5) with a nonsignificant trend towards decreased risk of diabetic nephropathy (OR=0.59). In almost all analyses, the burden test was more significant than either individual test. This increase in significance is expected if each variant is independently associated with the trait with the same effect direction.

TABLE 4A
SOS2 kidney associations

| | | | eGFR | | Creatinine | | BUN | |
|---|---|---|---|---|---|---|---|---|
| | SOS2 | | (n = 358,114) | | (n = 356,417) | | (n = 357,414) | |
| rsID | Direction | AAF | P value | Beta | P value | Beta | P value | Beta |
| rs72681869 | ↓ | 0.01 | 6.80E−14 | ↑0.072 | 1.74E−15 | ↓−0.006 | 1.27E−04 | ↓−0.004 |
| rs137961578 | ↓ | 0.001 | 0.022 | ↑0.060 | 0.0250 | ↓−0.005 | 0.176 | ↓−0.004 |
| Burden | ↓ | 0.01 | 4.22E−15 | ↑0.071 | 1.25E−16 | ↓−0.006 | 4.88E−05 | ↓−0.004 |

TABLE 4B
SOS2 kidney associations

| | Chronic Kidney | | Hypertensive CKD | | Diabetic | | Proteinuria | |
|---|---|---|---|---|---|---|---|---|
| | Disease (n = 12,863) | | (n = 1,412) | | Nephropathy (n = 548) | | (n = 650) | |
| rsID | P value | OR | P value | OR | P value | OR | P value | OR |
| rs72681869 | 0.01 | ↓0.857 | 0.128 | ↓0.716 | 0.188 | ↓0.579 | 0.023 | ↓0.422 |
| rs137961578 | 0.379 | ↓0.933 | 0.204 | ↓0.243 | 1.000 | ↓0.644 | 0.709 | ↑1.083 |
| Burden | 0.006 | ↓0.863 | 0.043 | ↓0.662 | 0.169 | ↓0.586 | 0.043 | ↓0.498 |

Pleiotropic associations were found for other diseases and traits. The rs72681869 (P191R) variant was significantly associated with decreased blood urate and decreased risk of gout, indicating that SOS2 LoF may protect against gout and hyperuricemia (Table 5). Both variants were also associated with reduced risk of hypertension and related traits (Table 6A and Table 6B). Both variants were also associated with decreased risk of cerebrovascular disease (Table 7). The rs72681869 (P191R) variant was also associated with decreased risk of type 2 diabetes (T2D) and related traits (Table 8A and Table 8B). Both variants were also associated with reduced risk of glaucoma and decreased intraocular pressure (Table 9A and Table 9B). Both variants were also associated with decreased liver enzymes levels and decreased risk of fatty liver disease (Table 10). Finally, both variants were associated with decreased risk on nonscarring hair loss (male pattern baldness) and increased blood testosterone levels in males (Table 11). In almost all analyses, the burden test was more significant than either individual test. This increase in significance is expected if each variant is independently associated with the trait with the same effect direction.

These protective associations with loss of function variants in SOS2 across several related and distinct diseases and traits indicate that inhibition of SOS2 may be therapeutic in at least these diseases or indications.

TABLE 5
SOS2 gout associations

| | | | Blood Urate | | Gout | | Medication | |
|---|---|---|---|---|---|---|---|---|
| | SOS2 | | (n = 357,600) | | (n = 13,246) | | Allopurinol (n = 4,230) | |
| rsID | Direction | AAF | P value | Beta | P value | OR | P value | OR |
| rs72681869 | ↓ | 0.01 | 1.96E−15 | ↓−0.009 | 5.06E−06 | ↓0.757 | 2.39E−04 | ↓0.656 |
| rs137961578 | ↓ | 0.001 | 0.015 | ↓−0.007 | 0.980 | ↓0.999 | 0.883 | ↑1.014 |
| Burden | ↓ | 0.01 | 1.20E−16 | ↓−0.009 | 1.86E−05 | ↓0.786 | 6.53E−04 | ↓0.697 |

TABLE 6A
SOS2 hypertension associations

| | | | Essential | | Systolic Blood Pressure | |
|---|---|---|---|---|---|---|
| | SOS2 | | Hypertension (n = 133,299) | | (n = 349,128) | |
| rsID | Direction | AAF | P value | OR | P value | Beta |
| rs72681869 | ↓ | 0.01 | 5.40E−10 | ↓0.922 | 1.29E−16 | ↓−0.006 |
| rs137961578 | ↓ | 0.001 | 0.098 | ↓0.952 | 0.083 | ↓−0.003 |
| Burden | ↓ | 0.01 | 1.09E−10 | ↓0.925 | 3.75E−17 | ↓−0.005 |

TABLE 6B
SOS2 hypertension associations

| | Diastolic Blood Pressure | | Mean Arterial Pressure | | Pulse Pressure | |
|---|---|---|---|---|---|---|
| | (n = 349,620) | | (n = 347,982) | | (n = 347,982) | |
| rsID | P value | Beta | P value | Beta | P value | Beta |
| rs72681869 | 5.40E−18 | ↓−0.006 | 1.02E−19 | ↓−1.375 | 7.79E−05 | ↓−0.005 |
| rs137961578 | 0.013 | ↓−0.005 | 0.023 | ↓−0.929 | 0.624 | ↓−0.002 |
| Burden | 2.30E−19 | ↓−0.006 | 8.05E−21 | ↓−1.332 | 8.47E−05 | ↓−0.004 |

TABLE 7
SOS2 cerebrovascular associations

| | | | Cerebrovascular Diseases | | Ischemic Cerebrovascular | |
|---|---|---|---|---|---|---|
| | SOS2 | | (n = 17,100) | | Disease (n = 11,825) | |
| rsID | Direction | AAF | P value | OR | P value | OR |
| rs72681869 | ↓ | 0.01 | 1.74E−05 | ↓0.801 | 0.012 | ↓0.878 |
| rs137961578 | ↓ | 0.001 | 0.001 | ↓0.583 | 0.006 | ↓0.543 |
| Burden | ↓ | 0.01 | 3.13E−07 | ↓0.777 | 0.001 | ↓0.840 |

TABLE 8A
SOS2 diabetes and metabolic associations

| | | | Type 2 Diabetes mellitus | | Diabetic Eye Disease | |
|---|---|---|---|---|---|---|
| | SOS2 | | (n = 25,321) | | (n = 6,613) | |
| rsID | Direction | AAF | P value | OR | P value | OR |
| rs72681869 | ↓ | 0.01 | 9.41E−06 | ↓0.825 | 0.004 | ↓0.759 |
| rs137961578 | ↓ | 0.001 | 0.191 | ↓0.874 | 0.639 | ↓0.856 |
| Burden | ↓ | 0.01 | 4.31E−06 | ↓0.831 | 0.002 | ↓0.764 |

TABLE 8B
SOS2 diabetes and metabolic associations

| | Medication | | Medication | | | |
|---|---|---|---|---|---|---|
| | Metformin | | insulin | | Hemoglobin A1C | |
| | (n = 9,262) | | (n = 3,663) | | (n = 320,384) | |
| rsID | P value | OR | P value | OR | P value | Beta |
| rs72681869 | 0.008 | ↓0.818 | 0.092 | ↓0.807 | 1.57E−10 | ↓−0.003 |
| rs137961578 | 0.968 | ↑1.028 | 0.209 | ↓0.581 | 0.236 | ↓−0.002 |
| Burden | 0.013 | ↓0.843 | 0.030 | ↓0.771 | 9.76E−11 | ↓−0.003 |

TABLE 9A
SOS2 glaucoma associations

| | | | | | Open-Angle | |
|---|---|---|---|---|---|---|
| | | | Glaucoma | | Glaucoma | |
| | SOS2 | | (n = 12,173) | | (n = 2,312) | |
| rsID | Direction | AAF | P value | OR | P value | OR |
| rs72681869 | ↓ | 0.01 | 4.77E−04 | ↓0.795 | 5.27E−04 | ↓0.519 |
| rs137961578 | ↓ | 0.001 | 0.510 | ↓0.921 | 0.018 | ↓0.152 |
| Burden | ↓ | 0.01 | 4.78E−04 | ↓0.810 | 5.38E−05 | ↓0.477 |

TABLE 9B
SOS2 glaucoma associations

| | Glaucoma Medications | | IOP (Goldmann- | |
|---|---|---|---|---|
| | (n = 3,575) | | correlated) (n = 79,800) | |
| rsID | P value | OR | P value | Beta |
| rs72681869 | 0.01 | ↓0.712 | 1.36E−04 | ↓−0.092 |
| rs137961578 | 0.114 | ↓0.491 | 0.095 | ↓−0.119 |
| Burden | 0.003 | ↓0.686 | 3.65E−05 | ↓−0.094 |

TABLE 10
SOS2 liver associations

| | | | ALT | | AST | | Non-Alcoholic Fatty | |
|---|---|---|---|---|---|---|---|---|
| | SOS2 | | (n = 356,653) | | (n = 355,516) | | Liver Disease (n = 4,129) | |
| rsID | Direction | AAF | P value | Beta | P value | Beta | P value | OR |
| rs72681869 | ↓ | 0.01 | 4.46E−20 | ↓−0.019 | 2.39E−26 | ↓−0.013 | 0.012 | ↓0.742 |
| rs137961578 | ↓ | 0.001 | 0.001 | ↓−0.018 | 2.05E−04 | ↓−0.013 | 0.040 | ↓0.423 |
| Burden | ↓ | 0.01 | 1.60E−22 | ↓−0.019 | 1.12E−29 | ↓−0.013 | 0.002 | ↓0.704 |

TABLE 11
SOS2 hair loss and testosterone associations

| | | | Nonscarring Hair | | Testosterone | |
|---|---|---|---|---|---|---|
| | | | Loss in | | in Males | |
| | SOS2 | | Males (n = 119,281) | | (n = 162,659) | |
| rsID | Direction | AAF | P value | OR | P value | Beta |
| rs72681869 | ↓ | 0.01 | 1.40E−06 | ↓0.938 | 2.93E−07 | ↑0.086 |
| rs137961578 | ↓ | 0.001 | 0.203 | ↓0.980 | 0.007 | ↑0.120 |
| Burden | ↓ | 0.01 | 6.20E−07 | ↓0.944 | 8.45E−09 | ↑0.091 |

### Example 2: siRNA-Mediated Knockdown of SOS2 in PODO/TERT256 Cell Line

siRNAs will be targeted to SOS2 mRNA that may downregulate levels of SOS2 mRNA leading to a decrease in RAC1 protein activation, when administered to the cultured immortalized human podocyte cell line PODO/TERT256 (Evercyte Cat. No. CHT-033-0256).

On Day 0, the PODO/TERT256 cells will be seeded at 150,000 cells/mL into a Falcon 24-well tissue culture plate (ThermoFisher Cat. No. 353047) at 0.5 mL per well.

On Day 1, the SOS2 siRNA and negative control siRNA master mixes will be prepared. The SOS2 siRNA master mix will contain 350 uL of Opti-MEM (ThermoFisher Cat. No. 4427037-s1288 Lot No. ASO2B02D) and 3.5 ul of a mixture of the two SOS2 siRNAs (10 uM stock). The negative control siRNA master mix will contain 350 uL of Opti-MEM and 3.5 ul of negative control siRNA (ThermoFisher Cat. No. 4390843, 10 uM stock). Next, 3 uL of TransIT-X2 (Mims Cat. No. MIR-6000) is added to each master mix. The mixes are incubated for 15 minutes to allow transfection complexes to form, then 51 ul of the appropriate master mix+TransIT-X2 is added to duplicate wells of PODO/TERT256 cells with a final siRNA concentration of 10 nM.

On Day 3, 48 hours post-transfection, duplicate wells will be lysed using the Cells-to-Ct kit according to the manufacturer's protocol (ThermoFisher Cat. No. 4399002) or protein lysis buffer containing protease and phosphatase inhibitors. For the Cells-to-Ct, cells are washed with 50 ul using cold 1×PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. Primary immunoblotting antibodies are: anti-GAPDH, anti-RAC1, anti-GTP-RAC1 (Sigma, MO) and anti-SOS2 (Abcam, Cambridge, UK).

A decrease in SOS2 mRNA expression in the PODO/TERT256 cells is expected after transfection with the SOS2 siRNAs compared to SOS2 mRNA levels in PODO/TERT256 cells transfected with the non-specific control siRNA 48 hours after transfection. There is an expected decrease in the amount of activated RAC1, measured by quantifying the amount of GTP bound RAC1 versus total RAC1 in wells containing PODO/TERT256 cells transfected with the SOS2 siRNAs relative to the amount of GTP bound RAC1 versus total RAC1 in wells containing PODO/TERT256 cells transfected with a non-specific control siRNA 48 hours after transfection. These results are expected to show that the SOS2 siRNAs elicit knockdown of SOS2 mRNA in PODO/TERT256 cells and that the decrease in SOS2 expression is correlated with a decrease in activated RAC1.

### Example 3: ASO-Mediated Knockdown of SOS2 in PODO/TERT256 Cell Line

ASOs will be targeted to SOS2 mRNA that may downregulate levels of SOS2 mRNA leading to a decrease in RAC1 protein activation, when administered to the cultured immortalized human podocyte cell line PODO/TERT256 (Evercyte Cat. No. CHT-033-0256).

On Day 0, the PODO/TERT256 cells will be seeded at 150,000 cells/mL into a Falcon 24-well tissue culture plate (ThermoFisher Cat. No. 353047) at 0.5 mL per well.

On Day 1, the SOS2 ASO and negative control ASO master mixes will be prepared. The SOS2 ASO master mix will contain 350 uL of Opti-MEM (ThermoFisher Cat. No. 4427037-s1288 Lot No. AS02B02D) and 3.5 ul of a mixture of the two SOS2 ASOs (10 uM stock). The negative control ASO master mix will contain 350 uL of Opti-MEM and 3.5 ul of negative control ASO (ThermoFisher Cat. No. 4390843, 10 uM stock). Next, 3 uL of TransIT-X2 (Mims Cat. No. MIR-6000) is added to each master mix. The mixes are incubated for 15 minutes to allow transfection complexes to form, then 51 ul of the appropriate master mix+TransIT-X2 is added to duplicate wells of PODO/TERT256 cells with a final ASO concentration of 10 nM.

On Day 3, 48 hours post transfection, duplicate wells will be lysed using the Cells-to-Ct kit according to the manufacturer's protocol (ThermoFisher Cat. No. 4399002) or protein lysis buffer containing protease and phosphatase inhibitors. For the Cells-to-Ct, cells are washed with 50 ul using cold 1×PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). For protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. Primary immunoblotting antibodies are: anti-GAPDH, anti-RAC1, anti-GTP-RAC1 (Sigma, MO) and anti-SOS2 (Abcam, Cambridge, UK).

A decrease in SOS2 mRNA expression in the PODO/TERT256 cells is expected after transfection with the SOS2 ASOs compared to SOS2 mRNA levels in PODO/TERT256 cells transfected with the non-specific control ASO 48 hours after transfection. There is an expected decrease in the amount of activated RAC1, measured by quantifying the amount of GTP bound RAC1 versus total RAC1 in wells containing PODO/TERT256 cells transfected with the SOS2 ASOs relative to the amount of GTP bound RAC1 versus total RAC1 in wells containing PODO/TERT256 cells transfected with a non-specific control ASO 48 hours after transfection. These results are expected to show that the SOS2 ASOs elicit knockdown of SOS2 mRNA in PODO/TERT256 cells and that the decrease in SOS2 expression is correlated with a decrease in activated RAC1.

### Example 4: Inhibition of SOS2 in a Mouse Model for Chronic Kidney Disease Using SOS2 siRNAs or ASOs

In this experiment, a mouse model of chronic kidney disease (CKD) will be used to evaluate the effect of siRNA or ASO inhibition of SOS2. The chronic kidney disease model involves temporarily occluding blood flow to both kidneys in 12-week-old C57BL mice. Kidney function is monitored by measuring serum creatinine, blood urea nitrogen, and urine albumin.

Mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group will include eight mice (4 males, 4 females).

Administration of siRNA or ASO will be achieved with a 200 ul subcutaneous injection of naked siRNA or ASO resuspended in PBS at concentration of 10 uM. On Study Day 0, Group 1 are be injected subcutaneously with non-targeting control siRNA, Group 2 mice are injected subcutaneously with non-targeting control ASO, Group 3 mice are injected subcutaneously with siRNA1 targeting mouse SOS2, Group 4 mice are injected subcutaneously with ASO1 targeting mouse SOS2, and Group 5 mice are injected subcutaneously with vehicle. Every 7 days after the first injection animals from each group are dosed for a total of 2 injections. Blood and urine samples are taken every other day and urine collected for a 24 hr period weekly; serum creatinine, blood urea nitrogen, blood uric acid and urine albumin are measured.

14 days after the ischemia reperfusion surgery to induce the chronic kidney disease like phenotype, the mice will be sacrificed by cervical dislocation following an intraperitoneal injection of 0.3 ml Nembutal (5 mg/ml) (Sigma Cat. No. 1507002). Final blood and urine samples are collected, and kidneys are removed and placed in RNAlater for mRNA isolation.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). A decrease in SOS2 mRNA expression in the kidney tissue from mice dosed with the SOS2 siRNA1 or ASO1 is expected compared to SOS2 mRNA levels in the kidney tissue from mice dosed with the non-specific controls. There is an expected decrease in serum creatinine, urea nitrogen, albumin, and urate in mice that receive the SOS2 siRNA or ASO compared to the serum creatinine, urea nitrogen, albumin, and urate in mice that receive the non-specific control. These results are expected to show that the SOS2 siRNA or ASO elicit knockdown of SOS2 mRNA in kidney tissue and that the decrease in SOS2 expression is correlated with a decrease in serum creatinine, blood urea nitrogen, and urine albumin.

### Example 5: Inhibition of SOS2 in a Mouse Model for Hyperuricemia-Induced Chronic Kidney Disease Using SOS2 siRNAs or ASOs

In this experiment, a mouse model of hyperuricemia-induced chronic kidney disease will be used to evaluate the effect of siRNA or ASO inhibition of SOS2. The hyperuricemia-induced chronic kidney disease model is induced by gavage of a mixture of adenine (160 mg/kg/d) and potassium oxonate (2400 mg/kg/d) (Sigma-Aldrich, St. Louis, MO) dissolved in 200 μL double distilled water daily consistently for 3 weeks in C57BL/6 mice (8-10 weeks of age weighing 25-27 g). Kidney function is monitored by measuring serum creatinine, blood urea nitrogen, blood uric acid and urine albumin.

3 hours after model induction, mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group contains eight mice (4 males, 4 females).

21 days after the model induction, the mice will be sacrificed by cervical dislocation following an intraperitoneal injection of 0.3 ml Nembutal (5 mg/ml) (Sigma Cat. No. 1507002). Final blood and urine samples are collected, and kidneys are removed and placed in RNAlater for mRNA isolation.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR was performed in triplicate using TaqMan Gene Expression Assays (Applied Biosystems FAM/SOS2 using a BioRad CFX96 Cat. No. 1855195). A decrease in SOS2 mRNA expression in the kidney tissue from mice dosed with the SOS2 siRNA1 or ASO1 is expected compared to SOS2 mRNA levels in the kidney tissue from mice dosed with the non-specific controls. There is an expected decrease in serum creatinine, blood urea nitrogen, blood uric acid and urine albumin in mice that receive the SOS2 siRNA or ASO compared to the serum creatinine, blood urea nitrogen, blood uric acid and urine albumin in mice that receive the non-specific control. These results are expected to show that the SOS2 siRNA or ASO elicit knockdown of SOS2 mRNA in kidney tissue and that the decrease in SOS2 expression is correlated with a decrease in serum creatinine, blood urea nitrogen, blood uric acid and urine albumin.

### Example 6: Inhibition of SOS2 in a Mouse Model of Glaucoma Using SOS2 siRNAs or ASOs

In this experiment, a mouse model of glaucoma will be used to evaluate the effect of siRNA or ASO inhibition of SOS2. The glaucoma model involves injection of adenovirus expressing TGF-B into the vitreous fluid of the eyes of C57BL mice. Glaucoma like conditions are monitored by measuring intra-ocular pressure (IOP) of the eyes of mice treated with the adenovirus expressing TGF-B.

Mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group contains eight mice (4 males, 4 females).

Administration of siRNA or ASO will be achieved with a 2 ul intra-vitreous injection of naked siRNA or ASO resuspended in PBS at concentration of 10 uM. On Study Day 0, Group 1 mice are injected subcutaneously with non-targeting control siRNA, Group 2 mice are injected subcutaneously with non-targeting control ASO, Group 3 mice are injected subcutaneously with siRNA1 targeting mouse SOS2, Group 4 mice are injected subcutaneously with ASO1 targeting mouse SOS2, and Group 5 mice are injected subcutaneously with vehicle. Every 7 days after the first injection animals from each group will be dosed for a total of 2 injections. IOP measurement are taken every other day.

14 days after the first injection of siRNA or ASO, the mice will be sacrificed by cervical dislocation following an intraperitoneal injection of 0.3 ml Nembutal (5 mg/ml) (Sigma Cat. No. 1507002). Eyes are removed and placed in RNAlater for mRNA isolation.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR was performed in triplicate using TaqMan Gene Expression Assays (Applied Biosystems FAM/SOS2 using a BioRad CFX96 Cat. No. 1855195). A decrease in SOS2 mRNA expression in the eye tissue from mice dosed with the SOS2 siRNA1 or ASO1 is expected compared to SOS2 mRNA levels in the eye tissue from mice dosed with the non-specific controls. There is an expected decrease in IOP in mice that receive the SOS2 siRNA or ASO compared to the IOP in mice that receive the non-specific control. These results are expected to show that the SOS2 siRNA or ASO elicit knockdown of SOS2 mRNA in eye tissue and that the decrease in SOS2 expression is correlated with a decrease in IOP.

### Example 7: Inhibition of SOS2 in a Mouse Model for NASH/NAFLD Using SOS2 siRNAs or ASOs

In this experiment, a mouse model of NASH/NAFLD will be used to evaluate the effect of siRNA or ASO inhibition of SOS2. The NASH/NAFLD disease model mice are fed a high fructose, high fat, high cholesterol diet in ob/ob mice for 12 weeks prior to treatment. Liver function is monitored by measuring non-fasted blood glucose, ALT, insulin, and lipids.

Mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group contains eight mice (4 males, 4 females).

Administration of siRNA or ASO will be achieved with a 200 ul subcutaneous injection of naked siRNA or ASO resuspended in PBS at concentration of 10 uM. On Study Day 0, Group 1 mice are injected subcutaneously with non-targeting control siRNA, Group 2 mice are injected subcutaneously with non-targeting control ASO, Group 3 mice are injected subcutaneously with siRNA1 targeting mouse SOS2, Group 4 mice are injected subcutaneously with ASO1 targeting mouse SOS2, and Group 5 mice are injected subcutaneously with vehicle. Every 7 days after the first injection animals from each group will be dosed for a total of 2 injections. Blood samples are taken twice per week; non-fasted blood glucose, ALT, insulin, and lipids are measured.

6 weeks after the treatment, the mice will be sacrificed by cervical dislocation following an intraperitoneal injection of 0.3 ml Nembutal (5 mg/ml) (Sigma Cat. No. 1507002). Final blood samples are collected, and livers are removed, and a section placed in RNAlater for mRNA isolation or neutral buffered formalin for histopathology.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). A decrease in SOS2 mRNA expression in the liver tissue from mice dosed with the SOS2 siRNA1 or ASO1 is expected compared to SOS2 mRNA levels in the liver tissue from mice dosed with the non-specific controls. There is an expected decrease in non-fasted blood glucose, ALT, insulin, and lipids in mice that receive the SOS2 siRNA or ASO compared to the non-fasted blood glucose, ALT, insulin, and lipids in mice that receive the non-specific control. These results are expected to show that the SOS2 siRNA or ASO elicit knockdown of SOS2 mRNA in liver tissue and that the decrease in SOS2 expression is correlated with a decrease in non-fasted blood glucose, ALT, insulin, and lipids.

Additionally, liver sections placed in the neutral buffered formalin will be embedded into paraffin and sectioned onto slide. The slide from both treated and non-treated mice are then assessed by histopathology using semi-quantitative scoring system (H&E/Picrosirius Red). There is an expected decrease in histopathology fatty liver and fibrosis scoring in mice that receive the SOS2 siRNA or ASO compared to the histopathology fatty liver and fibrosis scoring in mice that receive the non-specific control. These are expected to results show that the SOS2 siRNA or ASO elicit knockdown of SOS2 mRNA in liver tissue and that the decrease in SOS2 expression is correlated with a decrease in histopathology fatty liver and fibrosis scoring.

### IV. Sequence Information

Some embodiments include one or more nucleic acid sequences in the following tables:

### Sequence Information

| SEQ ID NO: | Description |
|---|---|
| 1 | Full-length human SOS2 mRNA sequence |
| | (Ensembl Acc.# ENST00000216373.10) |
| 2 | Modification pattern 1S |
| 3 | Modification pattern 2S |
| 4 | Modification pattern 3S |
| 5 | Modification pattern 4S |
| 6 | Modification pattern 5S |
| 7 | Modification pattern 1AS |
| 8 | Modification pattern 2AS |
| 9 | Modification pattern 3AS |
| 10 | Modification pattern 4AS |
| 11 | modification pattern ASO1 |

### Sequences

| SEQ ID | |
|---|---|
| NO: | 5′ to 3′ Sequence |
| 1 | AGAAGCGGGCCAGCGCCGCCGGGAAAGGAGGTCGCCGCCCGGGGTCGCCCGGGCT |
| | TGCGGGTCGCCGCGCTTCCTCTGCGGGCGCTGCGGGAGGCGAGGGCCGCCTCGGCG |
| | GAGGAAGCCGACCCCGGGGTCTCCGCCAGCCCGCCGGCGCCGCCTCCGAGCCGCCT |
| | CGGCCGTAGCCTCGGCCCGGGCGGGAGGCGGCGGCCGCCGGCGAGGCGAGGCGGC |
| | CCGCGCCCTGCCTGTCAGGCCACCGGCCCGGCCCGCGGGCTGCCCCCGATGCGGAG |
| | GCGCTGTCGCCGGGGCCATGCAGCAGGCGCCGCAGCCTTACGAGTTCTTCAGCGAG |
| | GAGAACAGTCCGAAATGGCGGGGACTGTTGGTCTCGGCCCTGCGGAAGGTTCAGGA |
| | ACAAGTGCATCCCACTCTCTCAGCTAATGAAGAGTCTCTCTATTATATTGAAGAGCT |
| | GATTTTTCAGCTGCTTAATAAATTATGCATGGCCCAGCCAAGGACTGTTCAAGATGT |
| | AGAGGAGCGAGTTCAGAAGACCTTTCCTCACCCAATTGATAAATGGGCCATTGCTG |
| | ATGCACAATCTGCTATAGAAAAACGAAAACGAAGAAATCCTCTTTTACTGCCTGTG |
| | GACAAAATCCATCCTTCGTTGAAGGAAGTATTAGGGTACAAAGTGGACTACCATGT |
| | ATCCCTATATATTGTGGCTGTACTAGAGTATATCTCAGCTGATATTTTAAAATTGGC |
| | TGGTAATTATGTTTTTAATATCCGGCATTATGAAATATCTCAGCAGGACATTAAAGT |
| | GTCAATGTGTGCGGATAAGGTTTTGATGGACATGTTTGATCAGGATGACATAGGTTT |
| | GGTTTCTCTCTGTGAAGATGAACCTAGTTCTTCTGGTGAATTAAACTACTATGATCT |
| | TGTCAGAACTGAAATCGCAGAAGAAAGACAGTATCTACGGGAATTAAATATGATCA |
| | TAAAAGTGTTTCGAGAAGCCTTTCTTTCTGATAGAAAGCTGTTTAAACCTTCTGATA |
| | TCGAAAAGATTTTTAGTAACATTTCAGATATACATGAATTGACTGTGAAACTTTTAG |
| | GTTTGATTGAAGACACAGTTGAAATGACTGATGAAAGCAGTCCTCATCCCTTAGCT |
| | GGCAGCTGTTTTGAAGATTTGGCAGAAGAGCAAGCATTTGATCCTTATGAAACATT |
| | ATCACAGGACATTCTTTCACCAGAGTTTCATGAACATTTCAATAAATTGATGGCCAG |
| | ACCTGCAGTTGCTCTACACTTTCAGTCCATTGCTGATGGTTTTAAAGAGGCAGTTCG |
| | TTATGTCCTTCCACGTCTTATGCTGGTGCCAGTGTATCACTGTTGGCACTACTTTGAG |
| | TTACTAAAGCAATTGAAAGCATGTAGTGAAGAACAAGAAGACAGAGAATGTTTGA |
| | ACCAAGCTATTACTGCTCTCATGAATCTCCAAGGTAGCATGGACCGAATTTACAAG |
| | CAGTATTCACCTAGACGTCGACCTGGAGATCCTGTTTGCCCTTTTTATAGTCACCAA |
| | TTAAGAAGCAAACACCTGGCTATCAAAAAAATGAATGAAATTCAGAAAAATATCG |
| | ATGGATGGGAAGGCAAAGATATTGGACAGTGTTGTAATGAATTCATTATGGAGGGA |
| | CCATTGACAAGAATCGGTGCCAAACATGAACGGCATATTTTTCTGTTTGATGGCTTA |
| | ATGATCAGTTGTAAACCTAATCATGGCCAGACTCGGCTTCCAGGTTACAGTAGTGC |
| | AGAATACAGGTTAAAAGAAAAATTTGTCATGAGGAAAATACAAATTTGTGATAAA |
| | GAAGATACTTGTGAGCACAAGCATGCATTTGAATTAGTATCCAAAGATGAGAACAG |
| | CATAATATTTGCTGCTAAGTCTGCTGAAGAAAAAAACAACTGGATGGCAGCCCTTA |
| | TTTCTCTTCATTATCGTAGTACTCTAGATCGAATGTTAGATTCAGTATTATTGAAAG |
| | AAGAAAATGAGCAACCACTGAGATTACCAAGTCCTGAAGTATATCGTTTTGTAGTA |
| | AAAGACTCTGAGGAAAACATTGTTTTTGAAGACAACTTGCAAAGTAGAAGTGGCAT |
| | CCCCATTATTAAAGGAGGAACTGTAGTGAAATTAATTGAAAGGTTAACATATCATA |
| | TGTATGCAGATCCCAATTTTGTTCGTACTTTTCTTACCACATATCGTTCATTTTGTAA |
| | ACCACAGGAATTGCTGAGCTTACTGATTGAACGGTTTGAAATTCCAGAGCCAGAAC |
| | CTACTGACGCAGACAAATTGGCAATAGAGAAAGGCGAGCAGCCAATCAGTGCAGA |
| | CCTTAAAAGATTTCGCAAGGAATATGTCCAACCAGTACAACTTAGGATCTTAAATG |
| | TATTTCGGCATTGGGTTGAACATCATTTTTATGACTTTGAAAGAGACTTGGAATTGC |
| | TTGAAAGACTAGAATCCTTCATTTCAAGTGTAAGAGGGAAAGCTATGAAAAAATGG |
| | GTAGAGTCAATTGCTAAGATCATCAGGAGGAAGAAGCAAGCTCAGGCAAACGGAG |
| | TAAGCCATAATATTACCTTTGAAAGTCCACCTCCACCAATTGAATGGCATATCAGCA |
| | AACCAGGACAGTTTGAAACATTTGATCTCATGACACTTCATCCAATAGAAATTGCA |
| | CGTCAGCTGACACTTTTGGAGTCTGATCTTTACAGGAAAGTTCAACCGTCTGAACTT |
| | GTAGGGAGTGTGTGGACCAAAGAAGATAAAGAAATAAATTCTCCAAATTTATTAAA |
| | AATGATTCGCCATACCACAAATCTCACCCTCTGGTTTGAAAAATGCATTGTGGAAG |
| | CAGAAAATTTTGAAGAACGGGTGGCAGTACTAAGTAGAATTATAGAAATTCTGCAA |
| | GTTTTTCAAGATTTGAATAATTTCAATGGCGTATTGGAGATAGTCAGTGCAGTAAAT |
| | TCAGTGTCAGTATACAGACTAGACCATACCTTTGAGGCACTGCAGGAAAGGAAAAG |
| | GAAAATTTTGGACGAAGCTGTGGAATTAAGTCAAGATCACTTTAAAAAATACCTAG |
| | TAAAACTTAAGTCAATCAATCCACCTTGTGTGCCTTTTTTTGGAATATATTTAACAA |
| | ATATTCTGAAGACCGAAGAAGGGAATAATGATTTTTTAAAAAAGAAAGGGAAAGA |
| | TTTAATCAATTTCAGTAAGAGGAGGAAAGTAGCTGAAATTACTGGAGAAATTCAGC |
| | AGTATCAGAATCAGCCTTACTGTTTACGGATAGAACCAGATATGAGGAGATTCTTT |
| | GAAAACCTTAACCCCATGGGAAGTGCATCTGAAAAAGAGTTTACAGATTATTTGTT |
| | CAACAAGTCACTAGAAATTGAACCTCGAAACTGCAAACAGCCACCTCGATTTCCTA |
| | GGAAATCAACTTTTTCCTTAAAATCTCCTGGAATAAGGCCTAACACAGGCCGACAT |
| | GGCTCTACCTCAGGTACTTTACGAGGTCACCCAACACCATTAGAAAGAGAACCATG |
| | TAAAATAAGCTTTAGTCGGATTGCTGAAACTGAGCTGGAATCAACAGTGTCAGCAC |
| | CAACCTCTCCAAATACACCATCTACTCCACCAGTATCTGCTTCTTCAGACCTTAGTG |
| | TATTTTTAGATGTGGATCTCAACAGCTCCTGTGGCAGCAATAGCATCTTTGCTCCAG |
| | TGCTTTTGCCACATTCAAAGTCTTTCTTTAGTTCATGTGGTAGTTTACATAAACTAAG |
| | TGAAGAGCCCCTGATTCCTCCTCCTCTTCCTCCTCGAAAAAAGTTTGATCATGATGC |
| | TTCAAATTCCAAGGGAAATATGAAATCTGATGATGATCCTCCTGCTATTCCACCGAG |
| | ACAGCCTCCTCCTCCAAAGGTAAAACCCAGAGTTCCTGTTCCTACTGGTGCATTTGA |
| | TGGGCCTCTGCATAGTCCACCTCCGCCACCACCAAGAGATCCTCTTCCTGATACCCC |
| | TCCACCAGTTCCCCTTCGGCCTCCAGAACACTTTATAAACTGTCCATTTAATCTTCA |
| | GCCACCTCCACTGGGGCATCTTCACAGAGATTCAGACTGGCTCAGAGACATTAGTA |
| | CGTGTCCAAATTCGCCAAGCACTCCTCCTAGCACACCCTCTCCAAGGGTACCGCGTC |
| | GATGCTATGTGCTCAGTTCTAGTCAGAATAATCTTGCTCATCCTCCAGCTCCCCCTG |
| | TTCCACCAAGGCAGAATTCAAGCCCTCATCTGCCAAAACTGCCACCAAAGACTTAC |
| | AAACGGGAGCTTTCGCACCCCCCATTGTACAGACTGCCTTTGCTAGAAAATGCAGA |
| | AACTCCCCAATGACCTTAGCCATATGTAGTCATTGACACTGGAATGGTATTTGTAAA |
| | GTTTTTTTTTTAATTTATTCAAAAAAAGACATAGTATTTTAGTACTTTTTACAAATAA |
| | TGCTCTTACAAAAATGCTACTGATCAAATAAGCTTTTAAGAATTGGAAAAATAAAA |
| | ATACAAAAGTCCTTCACCATTATCCTCAGGTGATCAGTAGCATTGCCTTGTCTTGGA |
| | TCCTCAGTGCTGCCAAAAGGCCAGTATAAAGAATTTATATTTGCACTGTAAACTCTG |
| | CAAAAATATGGTTTAAAGTGACATGATTGCACTGAAAAGGGATAGTGCTTTTGTGA |
| | AATTTTTCAAATTTGAGTAATAGATGCCTTTTTAAGGCAGTGAATTTACACAAATAT |
| | GGGAGGGGTATATGGTGTTACTGATTTTTAAACCTCTTTGACCATCTTCTAGTTTTTA |
| | CTTCTAGTTTTTACATCTAGGAGAATTGTGAATAACACCAACTGTTCTTAAACTCTT |
| | TAATGCCATGTCTTAAATGCCGGTATTTGCTGCTGAAGACAAAAATGAAAAGTAGG |
| | ATGAAAATAATAGAATGGCACTGTAAGTGTTTATTATTTTGTCAAAATGTAAACAA |
| | AGACTACATAACCCAATGATGGAGGGAAAAAGGGCATGTATCTCATTCAGATGTGC |
| | CTTTTGTTTTTGCAGACTATGACGTCTTTAGCTAATGAATTGCCTATTGTTATGGAAA |
| | ACAGTTAATATGCCATGTATGTACAGTTTTGTTTATATTGTATATTTAAAGATACTG |
| | CTAATAACCTATATAAATTTAAGTGACTTGAGGCCTATAATACAATCTGCTACTTTA |
| | CTAATTCATAAATTCAAAAAAAATTCTATGGCATAGGAACCAACTGCCTTGCCTTCA |
| | AGACCTAGTAACTTTCTCTATAAATCTCGTGTTAACTGAAATTTTTTTTAAATATATT |
| | TTTTAGATTGGTAATATTTAAACCAGCAAATACTTAAAGCTTTATTAAACATTTTAA |
| | TCAGATAAGTGAGTAAAGCTTTTATTTGCCATTTGGATGCCTTCGTTCAAAGTGATA |
| | GAGTGTTTTGCTGATAGTGCTGTAGCAGCAGTTGTAAAGTAGCCAAAAGCCACGTT |
| | GTTTATTTACTGGTTTGTGGCCTTTTACTGTGCTTTGTATCAGAGTTCTTAACAAGAT |
| | TAATAAATCACCCCAGTCTTAATTTTTAAAA |
| 2 | NfsnsNfnNfnNfNfNfnNfnNfnNfnNfnNfsnsn, where “Nf” is a 2′ fluoro-modified nucleoside, |
| | “n” is a 2′ O-methyl modified nucleoside, and “s” is a phosphorothioate linkage |
| 3 | nsnsnnNfnNfNfNfnnnnnnnnnnsnsn, where “Nf” is a 2′ fluoro-modified nucleoside, “n” is |
| | a 2′ O-methyl modified nucleoside, and “s” is a phosphorothioate linkage |
| 4 | nsnsnnNfnNfnNfnnnnnnnnnnsnsn, where “Nf” is a 2′ fluoro-modified nucleoside, “n” is |
| | a 2′ O-methyl modified nucleoside, and “s” is a phosphorothioate linkage |
| 5 | NfsnsNfnNfnNfNfNfnNfnNfnNfnNfnNfsnsnN-Lipid, where “Nf” is a 2′ fluoro-modified |
| | nucleoside, “n” is a 2′ O-methyl modified nucleoside, and “s” is a phosphorothioate |
| | linkage |
| 6 | nsnsnnNfnNfNfNfnnnnnnnnnnsnsnN-Lipid, where “Nf” is a 2′ fluoro-modified nucleoside, |
| | “n” is a 2′ O-methyl modified nucleoside, and “s” is a phosphorothioate linkage |
| 7 | nsNfsnNfnNfnNfnNfnnnNfnNfnNfnsnsn, where “Nf” is a 2′ fluoro-modified nucleoside, “n” |
| | is a 2′ O-methyl modified nucleoside, and “s” is a phosphorothioate linkage |
| 8 | nsNfsnnnNfnNfNfnnnnNfnNfnnnsnsn, where “Nf” is a 2′ fluoro-modified nucleoside, “n” |
| | is a 2′ O-methyl modified nucleoside, and “s” is a phosphorothioate linkage |
| 9 | nsNfsnnnNfnnnnnnnNfnNfnnnsnsn, where “Nf” is a 2′ fluoro-modified nucleoside, “n” |
| | is a 2′ O-methyl modified nucleoside, and “s” is a phosphorothioate linkage |
| 10 | nsNfsnNfnNfnnnnnnnNfnNfnnnsnsn, where “Nf” is a 2′ fluoro-modified nucleoside, “n” |
| | is a 2′ O-methyl modified nucleoside, and “s” is a phosphorothioate linkage |
| 11 | nsnsnsnsnsdNsdNsdNsdNsdNsdNsdNsdNsdNsdNsnsnsnsnsn, where “Nf” is a 2′ fluoro- |
| | modified nucleoside, “n” is a 2′ O-methyl modified nucleoside, and “s” is a |
| | phosphorothioate linkage |

1. A composition comprising an oligonucleotide that targets SOS2 and when administered to a subject in an effective amount increases an estimated glomerular filtration rate, or decreases a creatinine, blood urea nitrogen, proteinuria, microalbuminuria, blood urate, systolic or diastolic blood pressure, intraocular pressure, hemoglobin A1C, alanine aminotransferase, aspartate aminotransferase, or liver fat percentage measurement.

2. The composition of claim 1, wherein the estimated glomerular filtration rate is increased, or the creatinine, blood urea nitrogen, proteinuria, microalbuminuria, blood urate, systolic or diastolic blood pressure, intraocular, hemoglobin A1C, alanine aminotransferase, aspartate aminotransferase, or liver fat percentage measurement is decreased, by about 10% or more, as compared to prior to administration.

3-12. (canceled)

13. The composition of claim 1, wherein the oligonucleotide comprises a small interfering RNA (siRNA) comprising a sense strand and an antisense strand.

14. The composition of claim 13, wherein the sense strand is 12-30 nucleosides in length.

15. The composition of claim 13, wherein the antisense strand is 12-30 nucleosides in length.

16. A composition comprising an oligonucleotide that inhibits the expression of SOS2 wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 1.

17. The composition of claim 1, wherein the oligonucleotide comprises an antisense oligonucleotide (ASO).

18. The composition of claim 17, comprising the ASO that is complementary to a nucleoside sequence comprising about 12-30 contiguous nucleosides of SEQ ID NO: 1.

19. The composition of claim 17, wherein the ASO is 12-30 nucleosides in length.

20. The composition of claim 1, wherein the oligonucleotide comprises 1, 2, 3, 4, 5, 6, 7, 8, 9, 10, 11, 12, 13, 14, 15, 16, 17, 18, 19 or 20 modified internucleoside linkages.

21. The composition of claim 20, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

22. The composition of claim 20, wherein the modified internucleoside linkage comprises one or more phosphorothioate linkages.

23. (canceled)

24. The composition of claim 1, wherein the oligonucleotide comprises 1, 2, 3, 4, 5, 6, 7, 8, 9, 10, 11, 12, 13, 14, 15, 16, 17, 18, 19, or 20 modified nucleosides.

25. The composition of claim 24, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

26-27. (canceled)

28. The composition of claim 24, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

29. The composition of claim 24, wherein the modified nucleoside comprises one or more 2′fluoro modified nucleosides or 2′-O-alkyl modified nucleosides.

30. (canceled)

31. The composition of claim 24, wherein the oligonucleotide comprises a lipid attached at a 3′ or 5′ terminus of the oligonucleotide.

32. The composition of claim 31, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

33. (canceled)

34. The composition of claim 1, wherein the oligonucleotide comprises an N-acetylgalactosamine (GalNAc) ligand, an arginine-glycine-aspartic acid (RGD) peptide, or a cholesterol ligand.

35. A method of treating chronic kidney disease, diabetic nephropathy, gout, hyperuricemia, hypertension, cerebrovascular disease, type 2 diabetes, metabolic syndrome, obesity, glaucoma, non-alcoholic fatty liver disease, fibrotic liver disease, or hair loss in a subject in need thereof comprising administering to the subject a composition according to claim 1.
